SEQUENCE LISTING (24 sequences)

SEQ ID NO: 1 - DNA, 32 bp, Artificial Sequence
  Description of Artificial Sequence DNAenzyme
cgccattaggctagctacaacgacctagtgat

SEQ ID NO: 2 - DNA, 15 bp, Artificial Sequence
  Description of Artificial Sequenceantisense oligonucleotide
cttggccgctgccat

SEQ ID NO: 3 - DNA, 33 bp, Artificial Sequence
  Description of Artificial Sequence DNAenzyme
caggggacaggctagctacaacgacgttgcggg

SEQ ID NO: 4 - DNA, 15 bp, Artificial Sequence
  Description of Artificial Sequence antisense oligonucleotide
acacttttgtctgct

SEQ ID NO: 5 - DNA, 15 bp, Artificial Sequence
  Description of Artificial Sequence catalytic domain of DNAenzyme
ggctagctacaacga

SEQ ID NO: 6 - DNA, 33 bp, Artificial Sequence
  Description of Artificial Sequence DNAenzyme
tgcaggggaggctagctacaacgaaccgttgcg

SEQ ID NO: 7 - DNA, 33 bp, Artificial Sequence
  Description of Artificial Sequence DNAenzyme
catcctggaggctagctacaacgagagcaggct

SEQ ID NO: 8 - DNA, 33 bp, Artificial Sequence
  Description of Artificial Sequence DNAenzyme
ccgcggccaggctagctacaacgacctggacga

SEQ ID NO: 9 - DNA, 33 bp, Artificial Sequence
  Description of Artificial Sequence DNAenzyme
ccgctgccaggctagctacaacgacccggacgt

SEQ ID NO: 10 - DNA, 33 bp, Artificial Sequence
  Description of Artificial Sequence DNAenzyme
gcggggacaggctagctacaacgacagctgcat

SEQ ID NO: 11 - DNA, 33 bp, Artificial Sequence
  Description of Artificial Sequence DNAenzyme
cagcggggaggctagctacaacgaatcagctgc

SEQ ID NO: 12 - DNA, 33 bp, Artificial Sequence
  Description of Artificial Sequence DNAenzyme
ggtcagagaggctagctacaacgactgcagcgg

SEQ ID NO: 13 - DNA, 3068 bp, Mus musculus
ggggagccgccgccgcgattcgccgccgccgccagcttccgccgccgcaagatcggcccctgccccagcctccgcggcagccctgcgtccaccacgggccgcggctaccgccagcctgggggcccacctacactccccgcagtgtgcccctgcaccccgcatgtaacccggccaacccccggcgagtgtgccctcagtagcttcggccccgggctgcgcccaccacccaacatcagttctccagctcgctggtccgggarggcagcggccaaggccgagatgcaattgatgtctccgctgcagatctctgacccgttcggctcctttcctcactcacccaccatggacaactaccccaaactggaggagatgatgctgctgagcaacggggctccccagttcctcggtgctgccggaaccccagagggcagcggcggtaatagcagcagcagcaccagcagcgggggcggtggtgggggcggcagcaacagcggcagcagcgccttcaatcctcaaggggagccgagcgaacaaccctatgagcacctgaccacagagtccttttctgacatcgctctgaataatgagaaggcgatggtggagacgagttatcccagccaaacgactcggttgcctcccatcacctatactggccgcttctccctggagcccgcacccaacagtggcaacactttgtggcctgaaccccttttcagcctagtcagtggcctcgtgagcatgaccaatcctccgacctcttcatcctcggcgccttctccagctgcttcatcgtcttcctctgcctcccagagcccgcccctgagctgtgccgtgccgtccaacgacagcagtcccatctactcggctgcgcccacctttcctactcccaacactgacatttttcctgagccccaaagccaggcctttcctggctcggcaggcacagccttgcagtacccgcctcctgcctaccctgccaccaaaggtggtttccaggttcccatgatccctgactatctgtttccacaacaacagggagacctgagcctgggcaccccagaccagaagcccttccagggtctggagaaccgtacccagcagccttcgctcactccactatccactattaaagccttcgccactcagtcgggctcccaggacttaaaggctcttaataccacctaccaatcccagctcatcaaacccagccgcatgcgcaagtaccccaaccggcccagcaagacacccccccatgaacgcccatatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcgagaagccttttgcctgtgacatttgtgggaggaagtttgccaggagtgatgaacgcaagaggcataccaaaatccatttaagacagaaggacaagaaagcagacaaaagtgtggtggcctccccggctgcctcttcactctcttcttacccatccccagtggctacctcctacccatcccctgccaccacctcattcccatcccctgtgcccacttcctactcctctcctggctcctccacctacccatctcctgcgcacagtggcttcccgtcgccgtcagtggccaccacctttgcctccgttccacctgctttccccacccaggtcagcagcttcccgtctgcgggcgtcagcagctccttcagcacctcaactggtctttcagacatgacagcgaccttttctcccaggacaattgaaatttgctaaagggaataaaagaaagcaaagggagaggcaggaaagacataaaagcacaggagggaagagatggccgcaagaggggccacctcttaggtcagatggaagatctcagagccaagtccttctactcacgagtagaaggaccgttggccaacagccctttcacttaccatccctgcctcccccgtcctgttccctttgacttcagctgcctgaaacagccatgtccaagttcttcacctctatccaaaggacttgatttgcatggtattggataaatcatttcagtatcctctccatcacatgcctggcccttgctcccttcagcgctagaccatcaagttggcataaagaaaaaaaaatgggtttgggccctcagaaccctgccctgcatctttgtacagcatctgtgccatggattttgttttccttggggtattcttgatgtgaagataatttgcatactctattgtattatttggagttaaatcctcactttgggggaggggggagcaaagccaagcaaaccaatgatgatcctctattttgtgatgactctgctgtgacattaggtttgaagcattttttttttcaagcagcagtcctaggtattaactggagcatgtgtcagagtgttgttccgttaattttgtaaatactggctcgactgtaactctcacatgtgacaaagtatggtttgtttggttgggttttgtttttgagaatttttttgcccgtccctttggtttcaaaagtttcacgtcttggtgccttttgtgtgacacgccttccgatggcttgacatgcgcagatgtgagggacacgctcaccttagccttaagggggtaggagtgatgtgttgggggaggcttgagagcaaaaacgaggaagagggctgagctgagctttcggtctccagaatgtaagaagaaaaaatttaaacaaaaatctgaactctcaaaagtctatttttctaaactgaaaatgtaaatttatacatctattcaggagttggagtgttgtggttacctactgagtaggctgcagtttttgtatgttatgaacatgaagttcattattttgtggttttattttactttgtacttgtgtttgcttaaacaaagtaacctgtttggcttataaacacattgaatgcgctctattgcccatgggatatgtggtgtgtatccttcagaaaaattaaaaggaaaaat

SEQ ID NO: 14 - DNA, 4321 bp, Rattus rattus
ccgcggagcctcagctctacgcgcctggcgccctccctacgcgggcgtccccgactcccgcgcgcgttcaggctccgggttgggaaccaaggagggggagggtgggtgcgccgacccggaaacaccatataaggagcaggaaggatcccccgccggaacagaccttatttgggcagcgccttatatggagtggcccaatatggccctgccgcttccggctctgggaggaggggcgaacgggggttggggcgggggcaagctgggaactccaggagcctagcccgggaggccactgccgctgttccaatactaggctttccaggagcctgagcgctcagggtgccggagccggtcgcagggtggaagcgcccaccgctcttggatgggaggtcttcacgtcactccgggtcctcccggtcggtccttccatattagggcttcctgcttcccatatatggccatgtacgtcacggcggaggcgggcccgtgctgtttcagacccttgaaatagaggccgattcggggagtcgcgagagatcccagcgcgcagaacttggggagccgccgccgcgattcgccgccgccgccagcttccgccgccgcaagatcggcccctgccccagcctccgcggcagccctgcgtccaccacgggccgcggccaccgccagcctgggggcccacctacactccccgcagtgtgcccctgcaccccgcatgtaacccggccaacatccggcgagtgtgccctcagtagcttcggccccgggctgcgcccaccacccaacatcagctctccagctcgcacgtccgggatggcagcggccaaggccgagatgcaattgatgtctccgctgcagatctctgacccgttcggctcctttcctcactcacccaccatggacaactaccccaaactggaggagatgatgctgctgagcaacggggctccccagttcctcggtgctgccggaaccccagagggcagcggcggcaataacagcagcagcagcagcagcagcagcagcgggggcggtggtgggggcggcagcaacagcggcagcagcgctttcaatcctcaaggggagccgagcgaacaaccctacgagcacctgaccacaggtaagcggtggtctgcgccgaggctgaatcccccttcgtgactaccctaacgtccagtcctttgcagcacggacctgcatctagatcttagggacgggattgggatttccctctattccacacagctccagggacttgtgttagagggatgtctggggaccccccaaccctccatccttgcgggtgcgcggagggcagaccgtttgttttggatggagaactcaagttgcgtgggtggctggagtgggggagggtttgttttgatgagcagggttgccccctcccccgcgcgcgttgtcgcgagccttgtttgcagcttgttcccaaggaagggctgaaatctgtcaccagggatgtcccgccgcccagggtaggggcgcgcattagctgtggccactagggtgctggcgggattccctcaccccggacgcctgctgcggagcgctctcagagctgcagtagagggggattctctgtttgcgtcagctgtcgaaatggctctgccactggagcaggtccaggaacattgcaatctgctgctatcaattattaaccacatcgagagtcagtggtagccgggcgacctcttgcctggccgcttcggctctcatcgtccagtgattgctctccagtaaccaggcctctctgttctctttcctgccagagtccttttctgacatcgctctgaataacgagaaggcgctggtggagacaagttatcccagccaaactacccggttgcctcccatcacctatactggccgcttctccctggagcctgcacccaacagtggcaacactttgtggcctgaaccccttttcagcctagtcagtggccttgtgagcatgaccaaccctccaacctcttcatcctcagcgccttctccagctgcttcatcgtcttcctctgcctcccagagcccacccctgagctgtgccgtgccgtccaacgacagcagtcccatttactcagctgcacccacctttcctactcccaacactgacatttttcctgagccccaaagccaggcctgccaccaagggtggtttccaggttcccatgatccctgactatctgtttctttcctggctctgcaggcacagccttgcagtacccgcctcctgcctaccccacaacaacagggagacctgagcctgggcaccccagaccagaagcccttccagggtctggagaaccgtacccagcagccttcgctcactccactatccactatcaaagccttcgccactcagtcgggctcccaggacttaaaggctcttaataacacctaccagtcccaactcatcaaacccagccgcatgcgcaagtaccccaaccggcccagcaagacacccccccatgaacgcccgtatgcttgccctgttgagtcctgcgatcgccgcttttctcgctcggatgagcttacacgccacatccgcatccatacaggccagaagcccttccagtgtcgaatctgcatgcgtaatttcagtcgtagtgaccaccttaccacccacatccgcacccacacaggcgagaagccttttgcctgtgacatttgtgggagaaagtttgccaggagtgatgaacgcaagaggcataccaaaatccacttaagacagaaggacaagaaagcagacaaaagtgtcgtggcctcctcagctgcctcttccctctcttcctacccatccccagtggctacctcctacccatcccccgccaccacctcatttccatccccagtgcccacctcttactcctctccgggctcctctacctacccgtctcctgcacacagtggcttcccatcgccctcggtggccaccacctatgcctccgtcccacctgctttccctgcccaggtcagcaccttccagtctgcaggggtcagcaactccttcagcacctcaacgggtctttcagacatgacagcaaccttttctcctaggacaattgaaatttgctaaagggaatgaaagagagcaaagggaggggagcgcgagagacaataaaggacaggagggaagaaatggcccgcaagaggggctgcctcttaggtcagatggaagatctcagagccaagtccttctagtcagtagaaggcccgttggccaccagccctttcacttagcgtccctgccctccccagtcccggtccttttgacttcagctgcctgaaacagccacgtccaagttcttcacctctatccaaaggacttgatttgcatggtattggataaaccatttcagcatcatctccaccacatgcctggcccttgctcccttcagcactagaacatcaagttggctgaaaaaaaaaatgggtctgggccctcagaaccctgccctgtatctttgtacagcatctgtgccatggattttgttttccttggggtattcttgatgtgaagataatttgcatactctattgtactatttggagttaaattctcactttgggggagggggagcaaagccaagcaaaccaatggtgatcctctattttgtgatgatcctgctgtgacattaggtttgaaacttttttttttttttgaagcagcagtcctaggtattaactggagcatgtgtcagagtgttgttccgttaattttgtaaatactgctcgactgtaactctcacatgtgacaaaatacggtttgtttggttgggttttttgttgtttttgaaaaaaaaattttttttttgcccgtccctttggtttcaaaagtttcacgtcttggtgcctttgtgtgacacaccttgccgatggctggacatgtgcaatcgtgaggggacacgctcacctctagccttaagggggtaggagtgatgtttcaggggaggctttagagcacgatgaggaagagggctgagctgagctttggttctccagaatgtaagaagaaaaatttaaaacaaaaatctgaactctcaaaagtctatttttttaactgaaaatgtagatttatccatgttcgggagttggaatgctgcggttacctactgagtaggcggtgacttttgtatgctatgaacatgaagttcattattttgtggttttattttacttcgtacttgrgtttgcttaaacaaagtgacttgtttggcttataaacacattgaatgcgctttactgcccatgggatatgtggtgtgtatccttcagaaaaattaaaaggaaaataaagaaactaactggt

SEQ ID NO: 15 - DNA, 3132 bp, Homo sapiens
ccgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaacggtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtatcccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaaaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttcttttttttttttgaaagtgttttttcttcgtccttttggtttaaaaagtttcacgtcttggtgccttttgtgtgatgccccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 16 - DNA, 15 bp, Artificial Sequence
  Description of Artificial Sequence phosphorothioate-linked antisense oligonucleotide
cttggccgctgccat

SEQ ID NO: 17 - DNA, 15 bp, Artificial Sequence
  Description of Artificial Sequence phosphorothioate-linked antisense oligonucleotide
gcacttctgctgtcc

SEQ ID NO: 18 - DNA, 18 bp, Artificial Sequence
  Description of Artificial Sequence PCR primers
gcacccaacagtggcaac

SEQ ID NO: 19 - DNA, 18 bp, Artificial Sequence
  Description of Artificial Sequence PCR primers
gggatcatgggaacctgg

SEQ ID NO: 20 - DNA, 30 bp, Artificial Sequence
  Description of Artificial Sequence PCR primers
tgacggggtcacccacactgtgcccatcta

SEQ ID NO: 21 - DNA, 30 bp, Artificial Sequence
  Description of Artificial Sequence PCR primers
ctagaagcatttgcggtggacgatggaggg

SEQ ID NO: 22 - DNA, 19 bp, Rattus rattus
acguccgggauggcagcgg

SEQ ID NO: 23 - RNA, 19 nt, Homo sapiens
ucguccaggauggccgcgg

SEQ ID NO: 24 - DNA, 33 bp, Artificial Sequence
  Description of Artificial Sequence DNAenzyme
tcagctgcaggctagctacaacgactcggcctt

(19) United States
(12) Patent Application Publication
(10) Pub. No.: US 2003/0203864 A1
(43) Pub. Date: Oct. 30, 2003

(54) Treatment of cancer

(76) Inventor: Levon Michael Khachigian, Ryde (AU)

Correspondence Address: Gladys H. Monroy, Morrison & Foerster LLP, 755 Page Mill Road, Palo Alto, CA, 94304, US

(21) Appl. No.: 10/133,226

(22) Filed: Apr. 26, 2002

(51) Int. Cl. A61K 48/00; C12Q 1/68; G01N 33/574

(52) U.S. Cl. 514/44; 435/6; 435/7.23

(57) ABSTRACT

The present invention relates to a method for the treatment of tumours, the method comprising inhibiting angiogenesis in a subject in need thereof chracterised in that angigenesis in inhibited by administering to the subject an agent which inhibist induction of EGR, an agent which decreases the nuclerar accumulation or activity of EGR. The present invention also relates to a method of acreening for agents which inhibits angiogenesis.

### CROSS REFERENCE TO RELATED APPLICATIONS

This application claims priority from Australian application no. PQ 3676, filed Oct. 26, 1999 and PCT application no. PCT/AU00/01315, filed Oct. 26, 2000, the contents of each are incorporated herein by reference.

### FIELD OF THE INVENTION

The present invention relates to compositions and methods for the treatment of cancer.

### BACKGROUND OF THE INVENTION

Cancer

Cancer accounted for over half a million deaths in the United States in 1998 alone, or approximately 23% of all deaths (Landis et al. (1998), Cancer J. Clin. 48:6-29). Only cardiovascular disease consistently claims more lives (Cotran et al. (1999), Robbins pathologic basis of disease (6^{th} ed), W. B. Saunders, Philadelphia).

There is growing evidence that the cellular and molecular mechanisms underlying tumour growth involves more than just tumour cell proliferation and migration. Importantly, tumour growth and metastasis are critically dependent upon ongoing angiogenesis, the process of new blood vessel formation (Crystal, R. G., (1999), Cancer Chemother. Pharmacol. 43:S90-S99). Angiogenesis (also known as neovascularisation) is mediated by the migration and proliferation of vascular endothelial cells that sprout from existing blood vessels to form a growing network of microvessels that supply growing tumours with vital nutrients. Primary solid tumours cannot grow beyond 1-2 mm diameter without active angiogenesis (Harris, A. L. (1998), Recent Res. Cancer Res., 152:341-352).

Human HepG2 hepatocellular carcinoma cells have been used as a model cancer cell line for the assessment of anti-neoplastic drugs (Yang et al. (1997), Cancer Letters, 117:93-98). These cells basally and inducibly express the immediately-early gene and transcriptional regulator, early growth response factor-1 (EGR-1) (Kosaki et al. (1995), J. Biol. Chem., 270:20816-20823).

Early Growth Response Protein (EGR-1)

Early growth response factor-1 (EGR-1, also known as Egr-1, NGFI-A, zif268, krox24 and TIS8) is the product of an immediate early gene and a prototypical member of the zinc finger family of transcriptional regulators (Gashler, A. and Sukhatme, V. (1995), Prog. Nucl. Acid Res., 50:191-224). Egr-1 binds to the promoters of a spectrum of genes implicated in the pathogenesis of atherosclerosis and restenosis. These include the platelet-derived growth factor (PDGF) A-chain (Khachigian, L. M. et al. (1995), J. Biol. Chem., 270:27679-27686), PDGF-B (Khachigian, L. M. et al. (1996), Science, 271:1427-1431), transforming growth factor-β (Liu, C. et al. (1996), Proc. Natl. Acad. Sci. USA, 93:11831-11836 and Liu, C. et al. (1998), Cancer Gene Therapy, 5:3-28), fibroblast growth factor-2 (FGF-2) (Hu, R. M. and Levin, E. R. (1994), J. Clin. Invest., 93:1820-1827 and Biesiada, E. et al. (1996), J. Biol. Chem., 271:18576-18581), membrane type 1 matrix metalloproteinase (Haas, T. L., et al. (1999), J. Biol. Chem., 274:22679-22685), tissue factor (Cui, M. Z., et al. (1996), J. Biol. Chem., 271:2731-2739), and intercellular adhesion molecule-1 (Maltzman, J. S., et al. (1996), J. Exp. Med., 183:1747-1759). EGR-1 has also been localised to endothelial cells and smooth muscle cells in human atherosclerotic plaques (McCaffrey, T. A., et al. (2000), J. Clin. Invest., 105:653-662). Suppression of Egr-1 gene induction using sequence-specific catalytic DNA inhibits intimal thickening in the rat carotid artery following balloon angioplasty (Santiago, F. S., et al. (1999), Nature Med., 11:1264-1269).

DNAzymes

In human gene therapy, antisense nucleic acid technology has been one of the major tools of choice to inactivate genes whose expression causes disease and is thus undesirable. The anti-sense approach employs a nucleic acid molecule that is complementary to, and thereby hybridizes with, an mRNA molecule encoding an undesirable gene. Such hybridization leads to the inhibition of gene expression.

Anti-sense technology suffers from certain drawbacks. Anti-sense hybridization results in the formation of a DNA/target mRNA heteroduplex. This heteroduplex serves as a substrate for RNAse H-mediated degradation of the target mRNA component. Here, the DNA anti-sense molecule serves in a passive manner, in that it merely facilitates the required cleavage by endogenous RNAse H enzyme. This dependence on RNAse H confers limitations on the design of anti-sense molecules regarding their chemistry and ability to form stable heteroduplexes with their target mRNA's. Antisense DNA molecules also suffer from problems associated with non-specific activity and, at higher concentrations, even toxicity. An example of an alternative mechanism of antisense inhibition of target mRNA expression is steric inhibition of movement of the translational apparatus along the mRNA.

As an alternative to anti-sense molecules, catalytic nucleic acid molecules have shown promise as therapeutic agents for suppressing gene expression, and are widely discussed in the literature (Haseloff, J. and Gerlach, W. A. (1988), Nature, 334:585-591; Breaker (1994); Koizumi (1989); Otsuka; Kashani-Sabet (1992); Raillard (1996); and Carmi (1996)). Thus, unlike a conventional anti-sense molecule, a catalytic nucleic acid molecule functions by actually cleaving its target mRNA molecule instead of merely binding to it. Catalytic nucleic acid molecules can only cleave a target nucleic acid sequence if that target sequence meets certain minimum requirements. The target sequence must be complementary to the hybridizing arms of the catalytic nucleic acid, and the target must contain a specific sequence at the site of cleavage.

Catalytic RNA molecules (“ribozymes”) are well documented (Haseloff et al. (1988); Symonds (1992); and Sun (1997)), and have been shown to be capable of cleaving both RNA (Haseloff et al. (1988)) and DNA (Raillard (1996)) molecules. Indeed, the development of in vitro selection and evolution techniques has made it possible to obtain novel ribozymes against a known substrate, using either random variants of a known ribozyme or random-sequence RNA as a starting point (Pan (1992); Tsang (1994); and Breaker (1994)).

Ribozymes, however, are highly susceptible to enzymatic hydrolysis within the cells where they are intended to perform their function. This in turn limits their pharmaceutical applications.

Recently, a new class of catalytic molecules called “DNAzymes” was created (Breaker and Joyce (1995); Santoro (1997)). DNAzymes are single stranded, and cleave both RNA (Breaker (1994); Santoro (1997)) and DNA (Carmi (1996)). A general model for the DNAzyme has been proposed, and is known as the “10-23” model. DNAzymes following the “110-23” model, also referred to simply as “10-23 DNAzymes”, have a catalytic domain of 15 deoxyribonucleotides, flanked by two substrate-recognition domains of seven to nine deoxyribonucleotides each. In vitro analyses show that this type of DNAzyme can effectively cleave its substrate RNA at purine:pyrimidine junctions under physiological conditions (Santoro (1997)).

DNAzymes show promise as therapeutic agents. However, DNAzyme success against a disease caused by the presence of a known mRNA molecule is not predictable. This unpredictability is due, in part, to two factors. First, certain mRNA secondary structures can impede a DNAzyme's ability to bind to and cleave its target mRNA. Second, the uptake of a DNAzyme by cells expressing the target mRNA may not be efficient enough to permit therapeutically meaningful results.

### SUMMARY OF THE INVENTION

The present inventors have established that EGR-1 is critical in vascular endothelial cell replication and migration and that DNA-based, sequence-specific catalytic molecules targeting EGR-1 inhibit the growth of malignant cells in culture. These findings show that inhibitors of EGR or related EGR family members are useful in the treatment of tumours and that two separate mechanisms of action may involved. Specifically, inhibitors of EGR family members may inhibit tumour growth indirectly by inhibiting angiogenesis and/or directly by blocking the EGR family member in tumour cells.

When used herein the term “EGR” refers to a member of the EGR family. Members of the EGR family are described in Gashler et al., 1995 and include EGR-1 to EGR-4. It is currently preferred that the EGR family member is EGR-1.

Accordingly, in a first aspect the present invention provides a method for the treatment of a tumour, the method comprising administering to a subject in need thereof an agent which inhibits induction of EGR, an agent which decreases expression of EGR or an agent which decreases the nuclear accumulation or activity of EGR.

In a second aspect, the present invention provides a method for inhibiting the growth or proliferation of a tumour cell, the method comprising contacting a tumour cell with an agent which inhibits induction of EGR, an agent which decreases expression of EGR or an agent which decreases the nuclear accumulation or activity of EGR.

In a third aspect, the present invention provides a tumour cell which has been transformed by introducing into the cell a nucleic acid molecule, the nucleic acid molecule comprising or encoding (i) an agent which inhibits induction of EGR, (ii) an agent which decreases expression of EGR, or (iii) an agent which decreases the nuclear accumulation or activity of EGR.

In a fourth aspect, the present invention provides a method of screening for an agent which inhibits angiogenesis, the method comprising testing a putative agent for the ability to inhibit induction of EGR, decrease expression of EGR or decrease the nuclear accumulation or activity of EGR.

In a preferred embodiment of the present invention the agent is selected from the group consisting of an EGR antisense oligonucleotide, a ribozyme targeted against EGR, a ssDNA targeted against EGR dsDNA such that the ssDNA forms a triplex with the EGR-1 dsDNA, and a DNAzyme targeted against EGR.

### BRIEF DESCRIPTION OF THE FIGURES

FIG. 1. Insulin stimulates Egr-1-dependent gene expression in vascular endothelial cells. Growth-arrested bovine aortic endothelial cells previously transfected with pEBS1³foscat using FuGENE6 were incubated with D-glucose (5-30 mM), insulin (100 nM) or FGF-2 (25 ng/ml) as indicated for 24 h prior to preparation of cell lysates. CAT activity was normalized to the concentration of protein in the lysates.

FIG. 2. Insulin-induced DNA synthesis in aortic endothelial cells is blocked by antisense oligonucleotides targeting Egr-1. A, Insulin stimulates DNA synthesis. Growth-arrested endothelial cells were incubated with insulin (100 nM or 500 nM) or FBS (2.5%) for 18 h prior to ³H-thymidine pulse for a further 6 h. B, Antisense Egr-1 oligonucleotides inhibit insulin-inducible DNA synthesis. Endothelial cells were incubated with 0.8 μM of either AS2, AS2C or E3 prior to exposure to insulin (500 nM or 1000 nM) for 18 h and ³H-thymidine pulse for 6 h. C, Dose-dependent inhibition of insulin-inducible DNA synthesis. DNA synthesis stimulated by insulin (500 nM) was assessed in endothelial cells incubated with 0.4 μM or 0.8 μM of AS2 or AS2C. TCA-precipitable ³H-thymidine incorporation into DNA was assessed using a scintillation counter.

FIG. 3. Insulin-inducible DNA synthesis in cultured aortic endothelial cells is MEK/ERK-dependent. Growth quiescent endothelial cells were preincubated for 2 h with either PD98059 (10 μM or 30 μM), SB202190 (100 nM or 500 nM) or wortmannin (300 nM or 1000 nM) prior to the addition of insulin (500 nM) for 18 h and ³H-thymidine pulse. TCA-precipitable ³H-thymidine incorporation into DNA was assessed using a β-scintillation counter.

FIG. 4. Wound repair after endothelial injury is potentiated by insulin in an Egr-1 dependent manner. The population of cells in the denuded zone 3 d after injury in the various groups was quantitated and presented histodiagrammatically.

FIG. 5. Human microvascular endothelial cell proliferation is inhibited by DNA enzymes targeting human EGR-1. SV40-transformed HMEC-1 cells were grown in MCDB 131 medium with EGF (10 ng/ml) and hydrocortisone (1 μg/ml) supplements and 10% FBS. Forty-eight hours after incubation in serum-free medium without supplements, the cells were transfected with the indicated DNA enzyme (0.4 μM) and transfected again 72 h after the change of medium, when 10% serum was added. The cells were quantitated by Coulter counter, 24 h after the addition of serum.

FIG. 6. Sequence of NGFI-A DNAzyme (ED5), its scrambled control (ED5SCR) and 23 nt synthetic rat substrate. The translational start site is underlined.

FIG. 7. NGFI-A DNAzyme inhibits the induction of NGFI-A protein by serum (FBS). Western blot analysis was performed using antibodies to NGFI-A, Sp1 or c-Fos. The Coomassie Blue stained gel demonstrates that uniform amounts of protein were loaded per lane. The sequence of EDC is 5′-CGC CAT TAG GCT AGC TAC AAC GAC CTA GTG AT-3′ (SEQ ID NO:1); 3′T is inverted. SFM denotes serum-free medium.

FIG. 8. SMC proliferation is inhibited by NGFI-A DNAzyme. a, Assessment of total cell numbers by Coulter counter. Growth-arrested SMCs that had been exposed to serum and/or DNAzyme for 3 days were trypsinized followed by quantitation of the suspension. The sequence of AS2 is 5′-CTT GGC CGC TGC CAT-3′ (SEQ ID NO:2). b, Proportion of cells incorporating Trypan Blue after exposure to serum and/or DNAzyme. Cells were stained incubated in 0.2% (w:v) Trypan Blue at 22° C. for 5 min prior to quantitation by hemocytometer in a blind manner. c, Effect of ED5 on pup SMC proliferation. Growth-arrested WKY12-22 cells exposed to serum and/or DNAzyme for 3 days were resuspended and numbers were quantitated by Coulter counter. Data is representative of 2 independent experiments performed in triplicate. The mean and standard errors of the mean are indicated in the figure. * indicates P<0.05 (Student's paired t-test) as compared to control (FBS alone).

FIG. 9. NGFI-A DNAzyme inhibition of neointima formation in the rat carotid artery. A neointima was achieved 18 days after permanent ligation of the right common carotid artery. DNAzyme (500 μg) or vehicle alone was applied adventitially at the time of ligation and again after 3 days. Sequence-specific inhibition of neointima formation. Neointimal and medial areas of 5 consecutive sections per rat (5 rats per group) taken at 250 μm intervals from the point of ligation were determined digitally and expressed as a ratio per group. The mean and standard errors of the mean are indicated by the ordinate axis. * denotes P<0.05 as compared to the Lig, Lig+Veh or Lig+Veh+ED5SCR groups using the Wilcoxen rank sum test for unpaired data. Lig denotes ligation, Veh denotes vehicle.

FIG. 10. HepG2 cell proliferation is inhibited by 0.75 μM of DNAzyme DzA. Assessment of total cell numbers by Coulter counter. Growth-arrested cells that had been exposed to serum and/or DNAzyme for 3 days were trypsinized followed by quantitation of the suspension. The sequence of DzA is 5′caggggacaGGCTAGCTACAACGAcgttgcggg (SEQ ID NO:3).

### DETAILED DESCRIPTION OF THE INVENTION

In a first aspect the present invention provides a method for the treatment of a tumour, the method comprising administering to a subject in need thereof an agent which inhibits induction of an EGR, an agent which decreases expression of an EGR or an agent which decreases the nuclear accumulation or activity of an EGR.

The method of the first aspect may involve indirect inhibition of tumour growth by inhibiting angiogenesis and/or direct inhibition by blocking EGR in tumour cells.

In a preferred embodiment of the first aspect, the tumour is a solid tumour. The tumour may be selected from, without being limited to, a prostate tumour, a hepatocellular carcinoma, a skin carcinoma or a breast tumour.

As will be recognised by those skilled in this field there are a number means by which the method of the present invention may be achieved.

In a preferred embodiment of the present invention, the EGR is EGR-1.

In one embodiment, the method is achieved by targeting the EGR gene directly using triple helix (triplex) methods in which a ssDNA molecule can bind to the dsDNA and prevent transcription.

In another embodiment, the method is achieved by inhibiting transcription of the EGR gene using nucleic acid transcriptional decoys. Linear sequences can be designed that form a partial intramolecular duplex which encodes a binding site for a defined transcriptional factor. Evidence suggests that EGR transcription is dependent upon the binding of Sp1, AP1 or serum response factors to the promoter region. It is envisaged that inhibition of this binding of one or more of these transcription factors would inhibit transcription of the EGR gene.

In another embodiment, the method is achieved by inhibiting translation of the EGR mRNA using synthetic antisense DNA molecules that do not act as a substrate for RNase H and act by sterically blocking gene expression.

In another embodiment, the method is achieved by inhibiting translation of the EGR mRNA by destabilising the mRNA using synthetic antisense DNA molecules that act by directing the RNase H-mediated degradation of the EGR mRNA present in the heteroduplex formed between the antisense DNA and mRNA.

In one preferred embodiment of the present invention, the antisense oligonucleotide has a sequence selected from the group consisting of

(i) ACA CTT TTG TCT GCT (SEQ ID NO:4), and

(ii) CTT GGC CGC TGC CAT (SEQ ID NO:2).

In another embodiment, the method is achieved by inhibiting translation of the EGR mRNA by cleavage of the mRNA by sequence-specific hammerhead ribozymes and derivatives of the hammerhead ribozyme such as the Minizymes or Mini-ribozymes or where the ribozyme is derived from:

(i) the hairpin ribozyme,

(ii) the Tetrahymena Group I intron,

(iii) the Hepatitis Delta Viroid ribozyme or

(iv) the Neurospera ribozyme.

It will be appreciated by those skilled in the art that the composition of the ribozyme may be;

(i) made entirely of RNA,

(ii) made of RNA and DNA bases, or

(iii) made of RNA or DNA and modified bases, sugars and backbones.

Within the context of the present invention, the ribozyme may also be either;

(i) entirely synthetic or

(ii) contained within a transcript from a gene delivered within a virus derived vector, expression plasmid, a synthetic gene, homologously or heterologously integrated into the patients genome or delivered into cells ex vivo, prior to reintroduction of the cells of the patient, using one of the above methods.

In another embodiment, the method is achieved by inhibition of the ability of the EGR gene to bind to its target DNA by expression of an antisense EGR-1 mRNA.

In another embodiment, the method is achieved by inhibition of EGR activity as a transcription factor using transcriptional decoy methods.

In another embodiment, the method is achieved by inhibition of the ability of the EGR gene to bind to its target DNA by drugs that have preference for GC rich sequences. Such drugs include nogalamycin, hedamycin and chromomycin A3 (Chiang, et al., J. Biol. Chem (1996), 271:23999).

In a preferred embodiment, the method is achieved by cleavage of EGR mRNA by a sequence-specific DNAzyme. In a further preferred embodiment, the DNAzyme comprises:

(i) a catalytic domain which cleaves mRNA at a purine:pyrimidine cleavage site;

(ii) a first binding domain contiguous with the 5′ end of the catalytic domain; and

(iii) a second binding domain contiguous with the 3′ end of the catalytic domain,

wherein the binding domains are sufficiently complementary to two regions immediately flanking a purine:pyrimidine cleavage site within the region of EGR mRNA corresponding to nucleotides 168 to 332 as shown in SEQ ID NO:15, such that the DNAzyme cleaves the EGR mRNA.

As used herein, “DNAzyme” means a DNA molecule that specifically recognizes and cleaves a distinct target nucleic acid sequence, which may be either DNA or RNA.

In a preferred embodiment, the binding domains of the DNAzyme are complementary to the regions immediately flanking the cleavage site. It will be appreciated by those skilled in the art, however, that strict complementarity may not be required for the DNAzyme to bind to and cleave the EGR mRNA.

The binding domain lengths (also referred to herein as “arm lengths”) can be of any permutation, and can be the same or different. In a preferred embodiment, the binding domain lengths are at least 6 nucleotides. Preferably, both binding domains have a combined total length of at least 14 nucleotides. Various permutations in the length of the two binding domains, such as 7+7, 8+8 and 9+9, are envisioned.

The catalytic domain of a DNAzyme of the present invention may be any suitable catalytic domain. Examples of suitable catalytic domains are described in Santoro and Joyce, 1997 and U.S. Pat. No. 5,807,718. In a preferred embodiment, the catalytic domain has the nucleotide sequence GGCTAGCTACAACGA (SEQ ID NO:5).

Within the context of the present invention, preferred cleavage sites within the region of EGR mRNA corresponding to nucleotides 168 to 332 are as follows:

(i) the GU site corresponding to nucleotides 198-199;

(ii) the GU site corresponding to nucleotides 200-201;

(iii) the GU site corresponding to nucleotides 264-265;

(iv) the AU site corresponding to nucleotides 271-272;

(v) the AU site corresponding to nucleotides 292-293;

(vi) the AU site corresponding to nucleotides 301-302;

(vii) the GU site corresponding to nucleotides 303-304; and

(viii) the AU site corresponding to nucleotides 316-317.

In a further preferred embodiment, the DNAzyme has a sequence selected from:

(i) 5′-caggggacaGGCTAGCTACAACGAcgttgcggg (SEQ ID NO:3) targets GU (bp 198, 199); arms hybridise to bp 189-207

(ii) 5′-tgcaggggaGGCTAGCTACAACGAaccgttgcg (SEQ ID NO:6) targets GU (bp 200, 201); arms hybridise to bp 191-209

(iii) 5′-catcctggaGGCTAGCTACAACGAgagcaggct (SEQ ID NO:7) targets GU (bp 264 265); arms hybridise to bp 255-273

(iv) 5′-ccgcggccaGGCTAGCTACAACGAcctggacga (SEQ ID NO:8) targets AU (bp 271 272); arms hybridise to bp 262-280

(v) 5′-ccgctgccaGGCTAGCTACAACGAcccggacgt (SEQ ID NO:9) targets AU (bp 271 272); arms hybridise to bp 262-280

(vi) 5′-gcggggacaGGCTAGCTACAACGAcagctgcat (SEQ ID NO:10) targets AU (bp 301 302); arms hybridise to bp 292-310

(vii) 5′-cagcggggaGGCFAGCTACAACGAatcagctgc (SEQ ID NO:11) targets GU (bp 303, 304); arms hybridise to bp 294-312

(viii) 5′-ggtcagagaGGCTAGCTACAACGActgcagcgg (SEQ ID NO:12) targets AU (bp 316, 317); arms hybridise to bp 307-325.

In a particularly preferred embodiment, the DNAzyme targets the the GU site corresponding to nucleotides 198-199, the AU site corresponding to nucleotides 271-272 or the AU site corresponding to nucleotides 301-302.

In a further preferred embodiment, the DNAzyme has the sequence:

5′-caggggacaGGCTAGCFACAACGAcgttgcggg (SEQ ID NO:3),

5′-gcggggacaGGCTAGCTACAACcAcagctgcat (SEQ ID NO:10),

5′-ccgcggccaGGCTAGCTACAACGAcctggacga (SEQ ID NO:8) or

5′-ccgctgccaGGCTAGCTACAACGAcccggacgt (SEQ ID NO:9).

In applying DNAzyme-based treatments, it is preferable that the DNAzymes be as stable as possible against degradation in the intra-cellular milieu. One means of accomplishing this is by incorporating a 3′-3′ inversion at one or more termini of the DNAzyme. More specifically, a 3′-3′ inversion (also referred to herein simply as an “inversion”) means the covalent phosphate bonding between the 3′ carbons of the terminal nucleotide and its adjacent nucleotide. This type of bonding is opposed to the normal phosphate bonding between the 3′ and 5′ carbons of adjacent nucleotides, hence the term “inversion”. Accordingly, in a preferred embodiment, the 3′ end nucleotide residue is inverted in the building domain contiguous with the 3′ end of the catalytic domain. In addition to inversions, the instant DNAzymes may contain modified nucleotides. Modified nucleotides include, for example, N3′-P5′ phosphoramidate linkages, and peptide-nucleic acid linkages. These are well known in the art.

In a particularly preferred embodiment, the DNAzyme includes an inverted T at the 3′ position.

Although the subject may be any animal or human, it is preferred that the subject is a human.

Within the context of the present invention, the EGR inhibitory agents may be administered either alone or in combination with one or more additional anti-cancer agents which will be known to a person skilled in the art.

Administration of the inhibitory agents may be effected or performed using any of the various methods and delivery systems known to those skilled in the art. The administering can be performed, for example, intravenously, orally, via implant, transmucosally, transdermally, topically, intramuscularly, subcutaneously or extracorporeally. In addition, the instant pharmaceutical compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant composition. In one embodiment the delivery vehicle contains Mg²⁺ or other cation(s) to serve as co-factor(s) for efficient DNAzyme bioactivity.

Transdermal delivery systems include patches, gels, tapes and creams, and can contain excipients such as solubilizers, permeation enhancers (e.g., fatty acids, fatty acid esters, fatty alcohols and amino acids), hydrophilic polymers (e.g., polycarbophil and polyvinylpyrolidone), and adhesives and tackifiers (e.g., polyisobutylenes, silicone-based adhesives, acrylates and polybutene).

Transmucosal delivery systems include patches, tablets, suppositories, pessaries, gels and creams, and can contain excipients such as solubilizers; and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Topical delivery systems include, for example, gels and solutions, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In the preferred embodiment, the pharmaceutically acceptable carrier is a liposome or a biodegradable polymer. Examples of carriers which can be used in this invention include the following: (1) Fugene6® (Roche); (2) SUPERFECT® (Qiagen); (3) Lipofectamine 2000® (GIBCO BRL); (4) CellFectin, 1:1.5 (M/M) liposome formulation of the cationic lipid N,NI,NII,NIII-tetramethyl-N,NI,NII,NIII-tetrapalmitylspermine and dioleoyl phosphatidyl ethanolamine (DOPE)(GIBCO BRL); (5) Cytofectin GSV, 2:1 (M/M) liposome formulation of a cationic lipid and DOPE (Glen Research); (6) DOTAP (N-1(2,3-dioleoyloxy)-N,N,N-trimethyl-ammoniummethylsulfate) (Boehringer Manheim); and (7) Lipofectamine, 3:1 (M/M) liposome formulation of the polycationic lipid DOSPA and the neutral lipid DOPE (GIBCO BRL).

In a preferred embodiment, the agent is injected into or proximal the solid tumour. Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Delivery of the nucleic acid agents described may also be achieved via one or more, of the following non-limiting examples of vehicles:

(a) liposornes and liposome-protein conjugates and mixtures;

(b) non-liposomal lipid and cationic lipid formulations;

(c) activated dendrimer formulations;

(d) within polymer formulations such pluronic gels or within ethylene vinyl acetate coploymer (EVAc). The polymer may be delivered intra-luminally;

(e) within a viral-liposome complex, such as Sendai virus; or

(f) as a peptide-DNA conjugate.

Determining the prophylactically effective dose of the instant pharmaceutical composition can be done based on animal data using routine computational methods. In one embodiment, the prophylactically effective dose contains between about 0.1 mg and about 1 g of the instant DNAzyme. In another embodiment, the prophylactically effective dose contains between about 1 mg and about 100 mg of the instant DNAzyme. In a further embodiment, the prophylactically effective dose contains between about 10 mg and about 50 mg of the instant DNAzyme. In yet a further embodiment, the prophylactically effective dose contains about 25 mg of the instant DNAzyme.

It is also envisaged that nucleic acid agents targeting EGR may be administered by ex vivo transfection of cell suspensions, thereby inhibiting tumour growth, differentiation and/or metastasis.

In a second aspect, the present invention provides a method for inhibiting the growth or proliferation of a tumour cell, the method comprising contacting a tumour cell with an agent which inhibits induction of EGR, an agent which decreases expression of EGR or an agent which decreases the nuclear accumulation or activity of EGR.

In a third aspect, the present invention provides a tumour cell which has been transformed by introducing into the cell a nucleic acid molecule, the nucleic acid molecule comprising or encoding (i) an agent which inhibits induction of EGR, (ii) an agent which decreases expression of EGR, or (iii) an agent which decreases the nuclear accumulation or activity of EGR.

In a preferred embodiment of the third and fourth aspects, the agent is selected from the group consisting of an EGR antisense oligonucleotide or mRNA, a sequence-specific ribozyme targeted against EGR, a ssDNA targeted against EGR dsDNA and a sequence specific DNAzyme targeted against EGR.

In a fourth aspect, the present invention provides a method of screening for an agent which inhibits angiogenesis, the method comprising testing a putative agent for the ability to inhibit induction of EGR, decrease expression of EGR or decrease the nuclear accumulation or activity of EGR.

The putative agent may be tested for the ability to inhibit EGR by any suitable means. For example, the test may involve contacting a cell which expresses EGR with the putative agent and monitoring the production of EGR mRNA (by, for example, Northern blot analysis) or EGR protein (by, for example, immunahistochemical analysis or Western blot analysis). Other suitable tests will be known to those skilled in the art.

For reference, Table 1 below sets forth a comparison between the DNA sequences of mouse, rat and human EGR-1.

TABLE 1
Mouse, Rat and Human EGR-1

Symbol comparison table: GenRunData:pileupdna.cmp CampCheck: 6876
GapWeight: 5.000
GapLengthWeight: 0.300
EGR1align.msf MSF: 4388 Type: N Apr. 7, 1998 12:07 Check: 5107

| | |
|---|---|
| Name: mouseEGR1 | Len: 4388 Check: 8340 Weight: 1.00 (SEQ ID NO:13) |
| Name: ratEGR1 | Len: 4388 Check: 8587 Weight: 1.00 (SEQ ID NO:14) |
| Name: humanEGR1 | Len: 4388 Check: 8180 Weight: 1.00 (SEQ ID NO:15) |

NB. THIS IS RAT NGFI-A numbering

| | |
|---|---|
| | 1 50 |
| mouseEgr1 | .......... .......... .......... .......... .......... |
| ratNGFIA | CCGCGGAGCC TCAGCTCTAC GCGCCTGGCG CCCTCCCTAC GCGGGCGTCC |
| humanEGR1 | .......... .......... .......... .......... .......... |
| | 51 100 |
| mouseEGR1 | .......... .......... .......... .......... .......... |
| ratEGR1 | CCGACTCCCG CGCGCGTTCA GGCTCCGGGT TGGGAACCAA GGAGGGGGAG |
| humanEGR1 | .......... .......... .......... .......... .......... |
| | 101 150 |
| mouseEGR1 | .......... .......... .......... .......... .......... |
| ratEGR1 | GGTGGGTGCG CCGACCCGGA AACACCATAT AAGGAGCAGG AAGGATCCCC |
| humanEGR1 | .......... .......... .......... .......... .......... |
| | 151 200 |
| mouseEGR1 | .......... .......... .......... .......... .......... |
| ratEGR1 | CGCCGGAACA GACCTTATTT GGGCAGCGCC TTATATGGAG TGGCCCAATA |
| humanEGR1 | .......... .......... .......... .......... .......... |
| | 201 250 |
| mouseEGR1 | .......... .......... .......... .......... .......... |
| ratEGR1 | TGGCCCTGCC GCTTCCGGCT CTGGGAGGAG GGGCGAACGG GGGTTGGGGC |
| humanEGR1 | .......... .......... .......... .......... .......... |
| | 251 300 |
| mouseEGR1 | .......... .......... .......... .......... .......... |
| ratEGR1 | GGGGGCAAGC TGGGAACTCC AGGAGCCTAG CCCGGGAGGC CACTGCCGCT |
| humanEGR1 | .......... .......... .......... .......... .......... |
| | 301 350 |
| mouseEGR1 | .......... .......... .......... .......... .......... |
| ratEGR1 | GTTCCAATAC TAGGCTTTCC AGGAGCCTGA GCGCTCAGGG TGCCGGAGCC |
| humanEGR1 | .......... .......... .......... .......... .......... |
| | 351 400 |
| mouseEGR1 | .......... .......... .......... .......... .......... |
| ratEGR1 | GGTCGCAGGG TGGAAGCGCC CACCGCTCTT GGATGGGAGG TCTTCACGTC |
| humanEGR1 | .......... .......... .......... .......... .......... |
| | 401 450 |
| mouseEGR1 | .......... .......... .......... .......... .......... |
| ratEGR1 | ACTCCGGGTC CTCCCGGTCG GTCCTTCCAT ATTAGGGCTT CCTGCTTCCC |
| humanEGR1 | .......... .......... .......... .......... .......... |
| | 451 500 |
| mouseEGR1 | .......... .......... .......... .......... .......... |
| ratEGR1 | ATATATGGCC ATGTACGTCA CGGCGGAGGC GGGCCCGTGC TGTTTCAGAC |
| humanEGR1 | .......... .......... .......... .......... .......... |
| | 501 550 |
| mouseEGR1 | .......... .......... .......... .......... .......... |
| ratEGR1 | CCTTGAAATA GAGGCCGATT CGGGGAGTCG CGAGAGATCC CAGCGCGCAG |
| humanEGR1 | .......... .......... .......... .......... ....CCGCAG |
| | 551 600 |
| mouseEGR1 | .....GGGGA GCCGCCGCCG CGATTCGCCG CCGCCGCCAG CTTCCGCCGC |
| ratEGR1 | AACTTGGGGA GCCGCCGCCG CGATTCGCCG CCGCCGCCAG CTTCCGCCGC |
| humanEGR1 | AACTTGGGGA GCCGCCGCCG CCATCCGCCG CCGCAGCCAG CTTCCGCCGC |
| | 601 650 |
| mouseEGR1 | CGCAAGATCG GCCCCTGCCC CAGCCTCCGC GGCAGCCCTG CGTCCACCAC |
| rat EGR1 | CGCAAGATCG GCCCCTGCCC CAGCCTCCGC GGCAGCCCTG CGTCCACCAC |
| humanEGR1 | CGCAGGACCG GCCCCTGCCC CAGCCTCCGC AGCCGCGGCG CGTCCACGCC |
| | 651 700 |
| mouseEGR1 | GGGCCGCGGC TACCGCCAGC CTGGGGGCCC ACCTACACTC CCCGCAGTGT |
| ratEGR1 | GGGCCGCGGC CACCGCCAGC CTGGGGGCCC ACCTACACTC CCCGCAGTGT |
| humanEGR1 | CGCCCGCGCC CAGGGCGAGT CGGGGTCGCC GCCTGCACGC TTCTCAGTGT |
| | 701 750 |
| mouseEGR1 | GCCCCTGCAC CCCGCATGTA ACCCGGCCAA CCCCCGGCGA GTGTGCCCTC |
| ratEGR1 | GCCCCTGCAC CCCGCATGTA ACCCGGCCAA CATCCGGCGA GTGTGCCCTC |
| humanEGR1 | TCCCC.GCGC CCCGCATGTA ACCCGGCCAG GCCCCCGCAA CGGTGTCCCC |
| | 751 800 |
| mouseEGR1 | AGTAGCTTCG GCCCCGGGCT GCGCCCACC. .ACCCAACAT CAGTTCTCCA |
| ratEGR1 | AGTAGCTTCG GCCCCGGGCT GCGCCCACC. .ACCCAACAT CAGCTCTCCA |
| humanEGR1 | TGCAGCTCCA GCCCCGGGCT GCACCCCCCC GCCCCGACAC CAGCTCTCCA |
| | 801 850 |
| mouseEGR1 | GCTCGCTGGT CCGGGATGGC AGCGGCCAAG GCCGAGATGC AATTGATGTC |
| ratEGR1 | GCTCGCACGT CCGGGATGGC AGCGGCCAAG GCCGAGATGC AATTGATGTC |
| humanEGR1 | GCCTGCTCGT CCAGGATGGC CGCGGCCAAG GCCGAGATGC AGCTGATGTC |

ED5 (rat) arms hybridise to bp 807-825 in rat sequ
hED5(hum) arms hybridise to bp 262-280 in hum sequ

| | |
|---|---|
| | 851 900 |
| mouseEGR1 | TCCGCTGCAG ATCTCTGACC CGTTCGGCTC CTTTCCTCAC TCACCCACCA |
| ratEGR1 | TCCGCTGCAG ATCTCTGACC CGTTCGGCTC CTTTCCTCAC TCACCCACCA |
| humanEGR1 | CCCGCTGCAG ATCTCTGACC CGTTCGGATC CTTTCCTCAC TCGCCCACCA |
| | 901 950 |
| mouseEGR1 | TGGACAACTA CCCCAAACTG GAGGAGATGA TGCTGCTGAG CAACGGGGCT |
| ratEGR1 | TGGACAACTA CCCCAAACTG GAGGAGATGA TGCTGCTGAG CAACGGGGCT |
| humanEGR1 | TGGACAACTA CCCTAAGCTG GAGGAGATGA TGCTGCTGAG CAACGGGGCT |
| | 951 1000 |
| mouseEGR1 | CCCCAGTTCC TCGGTGCTGC CGGAACCCCA GAGGGCAGCG GCGGTAAT.. |
| ratEGR1 | CCCCAGTTCC TCGGTGCTGC CGGAACCCCA GAGGGCAGCG GCGGCAATAA |
| humanEGR1 | CCCCAGTTCC TCGGCGCCGC CGGGGCCCCA GAGGGCAGCG GCAGCAACAG |
| | 1001 1050 |
| mouseEGR1 | .......AGC AGCAGCAGCA CCAGCAGCGG GGGCGGTGGT GGGGGCGGCA |
| ratEGR1 | CAGCAGCAGC AGCAGCAGCA GCAGCAGCGG GGGCGGTGGT GGGGGCGGCA |
| humanEGR1 | CAGCAGCAGC AGCAGCGGGG GCGGTGGAGG CGGCGGGGGC GGCAGCAACA |
| | 1051 1100 |
| mouseEGR1 | GCAACAGCGG CAGCAGCGCC TTCAATCCTC AAGGGGAGCC GAGCGAACAA |
| ratEGR1 | GCAACAGCGG CAGCAGCGCT TTCAATCCTC AAGGGGAGCC GAGCGAACAA |
| humanEGR1 | GCAGCAGCAG CAGCAGCACC TTCAACCCTC AGGCGGACAC GGGCGAGCAG |
| | 1101 1150 |
| mouseEGR1 | CCCTATGAGC ACCTGACCAC AG...AGTCC TTTTCTGACA TCGCTCTGAA |
| ratEGR1 | CCCTACGAGC ACCTGACCAC AGGTAAGCGG TGGTCTGCGC CGAGGCTGAA |
| humanEGR1 | CCCTACGAGC ACCTGACCGC AG...AGTCT TTTCCTGACA TCTCTCTGAA |
| | 1151 1200 |
| mouseEGR1 | TAATGAGAAG GCGATGGTGG AGACGAGTTA TCCCAGCCAA ACGACTCGGT |
| ratEGR1 | TCCCCCTTCG TGACTACCCT AACGTCCAGT CCTTTGCAGC ACGGACCTGC |
| humanEGR1 | CAACGAGAAG GTGCTGGTGG AGACCAGTTA CCCCAGCCAA ACCACTCGAC |
| | 1201 1250 |
| mouseEGR1 | TGCCTCCCAT CACCTATACT GGCCGCTTCT CCCTGGAGCC CGCACCCAAC |
| ratEGR1 | ATCTAGATCT TAGGGACGGG ATTGGGATTT CCCTCTATTC ..CACACAGC |
| humanEGR1 | TGCCCCCCAT CACCTATACT GGCCGCTTTT CCCTGGAGCC TGCACCCAAC |
| | 1251 1300 |
| mousEGR1 | AGTGGCAACA CTTTGTGGCC TGAACCCCTT TTCAGCCTAG TCAGTGGCCT |
| ratEGR1 | TCCAGGGACT TGTGTTAGAG GGATGTCTGG GGACCCCCCA ACCCTCCATC |
| humanEGR1 | AGTGGCAACA CCTTGTGGCC CGAGCCCCTC TTCAGCTTGG TCAGTGGCCT |
| | 1301 1350 |
| mouseEGR1 | CGTGAGCATG ACCAATCCTC CGACCTCTTC ATCCTCGGCG CCTTCTCCAG |
| ratEGR1 | CTTGCGGGTG CGCGGAGGGC AGACCGTTTG TTTTGGATGG AGAACTCAAG |
| humanEGR1 | AGTGAGCATG ACCAACCCAC CGGCCTCCTC GTCCTCAGCA CCATCTCCAG |
| | 1351 1400 |
| mouseEGR1 | CTGCTTCATC GTCTTCCTCT GCCTCCCAGA GCCCGCCCCT GAGCTGTGCC |
| ratEGR1 | TTGCGTGGGT GGCT...... .....GGAGT GGGGGAGGGT TTGTTTTGAT |
| humanEGR1 | CGGCCTCCTC ......CTCC GCCTCCCAGA GCCCACCCCT GAGCTGCGCA |
| | 1401 1450 |
| mouseEGR1 | GTGCCGTCCA ACGACAGCAG TCCCATCTAC TCGGCTGCGC CCACCTTTCC |
| ratEGR1 | GAGCAGGGTT ......CCCC TCCCCCGCGC GCGTTGTCGC GAGCCTTGTT |
| humanEGR1 | GTGCCATCCA ACGACAGCAG TCCCATTTAC TCAGCGGCAC CCACCTTCCC |
| | 1451 1500 |
| mouseEGR1 | TACTCCCAAC ACTGACATTT TTCCTGAGCC CCAAAGCCAG GCCTTTCCTG |
| ratEGR1 | TGCAGCTTGT TCCCAAGGAA GGGCTGAAAT CTGTCACCAG GGATGTCCCG |
| humanEGR1 | CACGCCGAAC ACTGACATTT TCCCTGAGCC ACAAAGCCAG GCCTTCCCGG |
| | 1501 1550 |
| mouseEGR1 | GCTCGGCAGG CACAGCCTTG CAGTACCCGC CTCCTGCCTA CCCTGCCACC |
| ratEGR1 | CCGCCCAGGG TAGGGGCGCG CATTAGCTGT GGCC.ACTAG GGTGCTGGCG |
| humanEGR1 | GCTCGGCAGG GACAGCGCTC CAGTACCCGC CTCCTGCCTA CCCTGCCGCC |
| | 1551 1600 |
| mouseEGR1 | AAAGGTGGTT TCCAGGTTCC CATGATCCCT GACTATCTGT TTCCACAACA |
| ratEGR1 | GGATTCCCTC ACCCCGGACG CCTGCTGCGG AGCGCTCTCA GAGCTGCAGT |
| humanEGR1 | AAGGGTGGCT TCCAGGTTCC CATGATCCCC GACTACCTGT TTCCACAGCA |
| | 1601 1650 |
| mouseEGR1 | ACAGGGAGAC CTGAGCCTGG GCACCCCAGA CCAGAAGCCC TTCCAGGGTC |
| ratEGR1 | AGAGGGGGAT TCTCTGTTTG CGTCAGCTGT CGAAATGGCT CT......GC |
| humanEGR1 | GCAGGGGGAT CTGGGCCTGG GCACCCCAGA CCAGAAGCCC TTCCAGGGCC |
| | 1651 1700 |
| mouseEGR1 | TGGAGAACCG TACCCAGCAG CCTTCGCTCA CTCCACTATC CACTATTAAA |
| ratEGR1 | CACTGGAGCA GGTCCAGGAA CATTGCAATC TGCTGCTATC AATTATTAAC |
| humanEGR1 | TGGAGAGCCG CACCCAGCAG CCTTCGCTAA CCCCTCTGTC TACTATTAAG |
| | 1701 1750 |
| mouseEGR1 | GCCTTCGCCA CTCAGTCGGG CTCCCAGGAC TTAAAG.... ...GCTCTTA |
| ratEGR1 | CACATCGAGA GTCAGTGGTA GCCGGGCGAC CTCTTGCCTG GCCGCTTCGG |
| humanEGR1 | GCCTTTGCCA CTCAGTCGGG CTCCCAGGAC CTGAAG.... ..GCCCTCA |
| | 1751 1800 |
| mouseEGR1 | ATACCACCTA CCAATCCCAG CTCATCA..A ACCCAGCCCC ATGCGCAAGT |
| ratEGR1 | CTCTCATCGT CCAGTGATTG CTCTCCAGTA ACCAGGCCTC TCTGTTCTCT |
| humanEGR1 | ATACCAGCTA CCAGTCCCAG CTCATCA..A ACCCAGCCGC ATGCGCAAGT |
| | 1801 1850 |
| mouseEGR1 | ACCCCAACCG GCCCAGCAAG ACACCCCCCC ATGAACGCCC ATATGCTTGC |
| ratEGR1 | TTCCTGCCAG AGTCCTTTTC TGACATCGCT CTGAATAACG AGAAG..GCG |
| humanEGR1 | ATCCCAACCG GCCCAGCAAG ACGCCCCCCC ACGAACGCCC TTACGCTTGC |
| | 1851 1900 |
| mouseEGR1 | CCTGTCGAGT CCTGCGATCG CCGCTTTTCT CGCTCGGATG AGCTTACCCG |
| ratEGR1 | CTGGTGGAGA CAAGTTATCC CAGCCAAACT ACCCGGTTGC CTCCCATCAC |
| humanEGR1 | CCAGTGGAGT CCTGTGATCG CCGCTTCTCC CGCTCCGACG AGCTCACCCG |
| | 1901 1950 |
| mouseEGR1 | CCATATCCGC ATCCACACAG GCCAGAAGCC CTTCCAGTGT CGAATCTGCA |
| ratEGR1 | CTATACTGGC CGCTTCTCCC TGGAGCCTGC ACCCAACAGT GGCAACACTT |
| humanEGR1 | CCACATCCGC ATCCACACAG GCCAGAAGCC CTTCCAGTGC CGCATCTGCA |
| | 1951 2000 |
| mouseEGR1 | TGCGTAACTT CAGTCGTAGT GACCACCTTA CCACCCACAT CCGCACCCAC |
| ratEGR1 | TGTGGCCTGA ACCCCTTTTC AGCCTAGTCA GTGGCCTTGT GAGCATGACC |
| humanEGR1 | TGCGCAACTT CAGCCGCAGC GACCACCTCA CCACCCACAT CCGCACCCAC |
| | 2001 2050 |
| mouseEGR1 | ACAGGCGAGA AGCCTTTTGC CTGTGACATT TGTGGGAGGA AGTTTGCCAG |
| ratEGR1 | AACCCTCCAA CCTCTTCATC CTCAGCGCCT TCTCCAGCTG CTTCATCGTC |
| humanEGR1 | ACAGGCGAAA AGCCCTTCGC CTGCGACATC TGTGGAAGAA AGTTTGCCAG |
| | 2051 2100 |
| mouseEGR1 | GAGTGATGAA CGCAAGAGGC ATACCAAAAT CCATTTAAGA CAGAAGGACA |
| ratEGR1 | TTCCTCTGCC TCCCAGAGCC CACCCCTGAG CTGTGCCGTG CCGTCCAACG |
| humanEGR1 | GAGCGATGAA CGCAAGAGGC ATACCAAGAT CCACTTGCGG CAGAAGGACA |
| | 2101 2150 |
| mouseEGR1 | AGAAAGCAGA CAAAAGTGTG GTGGCCTCCC CGGCTGC... .CTCTTCACT |
| ratEGR1 | ACAGCAGTCC CATTTACTCA GCTGCACCCA CCTTTCCTAC TCCCAACACT |
| humanEGR1 | AGAAAGCAGA CAAAAGTGTT GTGGCCTCTT CGGCCACCTC CTCTCTCTCT |
| | 2151 2200 |
| mouseEGR1 | .......... .......... CTCTTCTTAC CCATCCCCAG TGGCTACCTC |
| ratEGR1 | .......... .......... GACATTTTTC CTGAGCCCCA AAGCCAGGCC |
| humanEGR1 | TCCTACCCGT CCCCGGTTGC TACCTCTTAC CCGTCCCCGG TTACTACCTC |
| | 2201 2250 |
| mouseEGR1 | CTACCCATCC CCTGCCACCA CCTCATTCCC ATCCCCTGTG CCCACTTCCT |
| ratEGR1 | TTTCCTGGCT CTGCAGGCAC AGCCTTGCAG TACCCGCCTC CTGCCTACCC |
| humanEGR1 | TTATCCATCC CCGGCCACCA CCTCATACCC ATCCCCTGTG CCCACCTCCT |
| | 2251 2300 |
| mouseEGR1 | ACTCCTCTCC TGGCTCCTCC ACCTACCCAT CTCCTGCGCA CAGTGGCTTC |
| ratEGR1 | TGCCACCAAG GGTGGTTTCC AGGTTCCCAT GATCCCTGAC TATCTGTTTC |
| humanEGR1 | TCTCCTCTCC CGGCTCCTCG ACCTACCCAT CCCCTGTGCA CAGTGGCTTC |
| | 2301 2350 |
| mouseEGR1 | CCGTCGCCGT CAGTGGCCAC CACCTTTGCC TCCGTTCC.. .......... |
| ratEGR1 | CACAACAACA GGGAGACCTG AGCCTGGGCA CCCCAGACCA GAAGCCCTTC |
| humanEGR1 | CCCTCCCCGT CGGTGGCCAC CACGTACTCC TCTGTTCCC. .......... |
| | 2351 2400 |
| mouseEGR1 | ....ACCTGC TTTCCCCACC CAGGTCAGCA GCTTCCCGTC TGCGGGCGTC |
| ratEGR1 | CAGGGTCTGG AGAACCGTAC CCAGCAGCCT TCGCTCACTC CACTATCCAC |
| humanEGR1 | .....CCTGC TTTCCCGGCC CAGGTCAGCA GCTTCCCTTC CTCAGCTGTC |
| | 2401 2450 |
| mouseEGR1 | AGCAGCTCCT TCAGCACCTC AACTGGTCTT TCAGACATGA CAGCGACCTT |
| ratEGR1 | TATCAAAGCC TTCGCCACTC AGTCGGGCTC CCAGGACTTA AAGGCTCTTA |
| humanEGR1 | ACCAACTCCT TCAGCGCCTC CACAGGGCTT TCGGACATGA CAGCAACCTT |
| | 2451 2500 |
| mouseEGR1 | TTCTCCCAGG ACAATTGAAA TTTGCTAAAG GGA....... .ATAAAAG.. |
| ratEGR1 | ATAACACCTA CCAGTCCCAA CTCATCAAAC CCAGCCGCAT GCGCAAGT.. |
| humanEGR1 | TTCTCCCAGG ACAATTGAAA TTTGCTAAAG GGAAAGGGGA AAGAAAGGGA |
| | 2501 2550 |
| mouseEGR1 | .AAAGCAAAG GGAGAGGCAG GAAAGACATA AAAGCA...C AGGAGGGAAG |
| ratEGR1 | .ACCCCAACC GGCCCAGCAA GACACCCCCC CATGAACGCC CGTATGCTTG |
| humanEGR1 | AAAGGGAGAA AAAGAAACAC AAGAGACTTA AAGGACAGGA GGAGGAGATG |
| | 2551 2600 |
| mouseEGR1 | AGATGGCCGC AAGAGGGGCC ACCTCTTAGG TCAGATGGAA GATCTCAGAG |
| ratEGR1 | CCCTGTTGAG TCCTGCGATC GCCGCTTTTC TCGCTCGGAT GAGCTTACAC |
| humanEGR1 | GCCATAGGAG AGGAGGGTT. .CCTCTTAGG TCAGATGGAG GTTCTCAGAG |
| | 2601 2650 |
| mouseEGR1 | CCAAGTCCTT CTACTCACGA GTA. . GAAGG ACCGTTGGCC AACAGCCCTT |
| ratEGR1 | GCCACATCCG CATCCATACA GGC. .CAGAA GCCCTTCCAG TGTCGAATCT |
| humanEGR1 | CCAAGTCCTC CCTCTCTACT GGAGTGGAAG GTCTATTGGC CAACAATCCT |
| | 2651 2700 |
| mouseEGR1 | TCACTTACCA TCCCTGCCTC CCCCGTCCTG TTCCCTTTGA CTTCAGCTGC |
| ratEGR1 | GCATGCGTAA TTTCAGTCGT AGTGACCACC TTACCACCCA CATCCGCACC |
| humanEGR1 | TTCTGCCCAC TTCCCCTTCC CCAATTACTA TTCCCTTTGA CTTCAGCTGC |
| | 2701 2750 |
| mouseEGR1 | CTGAAACAGC CATGTCCAAG TTCTTCACCT CTATCCAAAG GACTTGATTT |
| ratEGR1 | C..ACACAGG CGAGAAGCCT TTTGCCTGTG ACATTTGTGG GAGAAAGTTT |
| humanEGR1 | CTGAAACAGC CATGTCCAAG TTCTTCACCT CTATCCAAAG AACTTGATTT |
| | 2751 2800 |
| mouseEGR1 | GCATGG.... ..TATTGGAT AAATCATTTC AGTATCCTCT .......... |
| ratEGR1 | GCCAGGAGTG ATGAACGCAA GAGGCATACC AAAATCCACT TAAGACAGAA |
| humanEGR1 | GCATGGA... ..TTTTGGAT AAATCATTTC AGTATCATCT .......... |
| | 2801 2850 |
| mouseEGR1 | .....CCATC ACATGCCTGG CCCTTGCTCC CTTCAGCGCT AGACCATCAA |
| ratEGR1 | GGACAAGAAA GCAGACAAAA GTGTCGTGGC CTCCTCAGCT GCCTCTTCCC |
| humanEGR1 | ....CCATCA TATGCCTGAC CCCTTGCTCC CTTCAATGCT AGAAAATCGA |
| | 2851 2900 |
| mouseEGR1 | GTTGGCATAA AGAAAAAAAA ATGGGTTTGG GCCCTCAGAA CCCTGCCCTG |
| ratEGR1 | TCTCTTCCTA CCCATCCCCA GTGGCTACCT CCTACCCATC CCCCGCCACC |
| humanEGR1 | GTTGGC.... .....AAAAT GGGGTTTGGG CCCCTCAGAG CCCTGCCCTG |
| | 2901 2950 |
| mouseEGR1 | CATCTTTGTA CAGCATCTGT GCCATGGATT TTGTTTTCCT TGGGGTATTC |
| ratEGR1 | ACCTCATTTC CATCCCCAGT GCCCACCTCT TACTCCTCTC CGGGCTCCTC |
| humanEGR1 | CACCCTTGTA CAGTGTCTGT GCCATGGATT TCGTTTTTCT TGGGGTACTC |
| | 2951 3000 |
| mouseEGR1 | TTGATGTGAA GATAATTTGC ATACT..... .CTATTGTAT TATTTGGAGT |
| ratEGR1 | TACCTACCCG TCTCCTGCAC ACAGTGGCTT CCCATCGCCC TCGGTGGCCA |
| humanEGR1 | TTGATGTGAA GATAATTTGC ATATT..... .CTATTGTAT TATTTGGAGT |
| | 3001 3050 |
| mouseEGR1 | TAAATCCTCA CTTTGGGG.. GAGGGGGGAG CAAAGCCAAG CAAACCAATG |
| ratEGR1 | CCACCTATGC CTCCGTCC.. CACCTGCTTT CCCTGCCCAG GTCAGCACCT |
| humanEGR1 | TAGGTCCTCA CTTGGGGGAA AAAAAAAAAA AAAAGCCAAG CAAACCAATG |
| | 3051 3100 |
| mouseEGR1 | ATGATCCTCT ATTTTGTGAT GACTCTGCTG TGACATTA.. .......... |
| ratEGR1 | TCCAGTCTGC AGGGGTCAGC AACTCCTTCA GCACCTCAAC GGGTCTTTCA |
| humanEGR1 | GTGATCCTCT ATTTTGTGAT GATGCTGTGA CAATA..... .......... |
| | 3101 3150 |
| mouseEGR1 | .GGTTTGAAG CATTTTTTTT TTCAAGCAGC AGTCCTAGGT ATTAACTGGA |
| ratEGR1 | GACATGACAG CAACCTTTTC TCCTAGGACA ATTGAAATTT GCTAAAGGGA |
| humanEGR1 | ...AGTTTGA ACCTTTTTTT TTGAAACAGC AGTCCCAG.. ..TATTCTCA |
| | 3151 3200 |
| mouseEGR1 | ..GCATGTGT CAGAGTGTTG TTCCGTTAAT TTTGTAAATA CTGGCTCGAC |
| ratEGR1 | ATGAAAGAGA GCAAAGGGAG GGGAGCGCGA GAGACAATAA AGGACAGGAG |
| humanEGR1 | GAGCATGTGT CAGAGTGTTG TTCCGTTAAC CTTTTTGTAA ATACTGCTTG |
| | 3201 3250 |
| mouseEGR1 | .TGTAACTCT CACATGTGAC AAAGTATGGT TTGTTTGGTT GGGTTTTGTT |
| ratEGR1 | .GGAAGAAAT GGCCCGCAAG AGGGGCTGCC TCTTAGGTCA GATGGAAGAT |
| humanEGR1 | ACCGTACTCT CACATGTGGC AAAATATGGT TTGGTTTTTC TTTTTTTTTT |
| | 3251 3300 |
| mouseEGR1 | TTTGAGAATT TTTTTGCCCG TCCCTTTGGT TTCAAAAGTT TCACGTCTTG |
| ratEGR1 | CTCAGAGCCA AGTCCTTCTA GTCAGTAGAA GGCCCGTTGG CCACCAGCCC |
| humanEGR1 | TTGAAAGTGT TTTTTCTTCG TCCTTTTGGT TTAAAAAGTT TCACGTCTTG |
| | 3301 3350 |
| mouseEGR1 | GTGCCTTTTG TGTGACACGC CTT.CCGATG GCTTGACATG CGCA...... |
| ratEGR1 | TTTCACTTAG CGTCCCTGCC CTC.CCCAGT CCCGGTCCTT TTGACTTCAG |
| humanEGR1 | GTGCCTTTTG TGTGATGCCC CTTGCTGATG GCTTGACATG TGCAAT.... |
| | 3351 3400 |
| mouseEGR1 | ...GATGTGA GGGACACGCT CACCTTAGCC TTAA...GGG GGTAGGAGTG |
| ratEGR1 | CTGCCTGAAA CAGCCACGTC CAAGTTCTTC ACCT...CTA TCCAAAGGAC |
| humanEGR1 | .....TGTGA GGGACATGCT CACCTCTAGC CTTAAGGGGG GCAGGGAGTG |
| | 3401 3450 |
| mouseEGR1 | ATGTGTTGGG GGAGGCTTGA GAGCAAAAAC GAGGAAGAGG GCTGAGCTGA |
| ratEGR1 | TTGATTTGCA TGGTATTGGA TAAACCATTT CAGCATCATC TCCACCACAT |
| humanEGR1 | ATGATTTGGG GGAGGCTTTG GGAGCAAAAT AAGGAAGAGG GCTGAGCTGA |
| | 3451 3500 |
| mouseEGR1 | GCTTTCGGTC TCCAGAATGT AAGAAGAAAA AATTTAAACA AAAATCTGAA |
| ratEGR1 | GCCTGGCCCT TGCTCCCTTC AGCACTAGAA CATCAAGTTG GCTGAAAAAA |
| humanEGR1 | GCTTCGGTTC TCCAGAATGT AAGAAAACAA AATCTAAAAC AAAATCTGAA |
| | 3501 3550 |
| mouseEGR1 | CTCTCAAAAG TCTATTTTTC TAAACTGAAA ATGTAAATTT ATACATCTAT |
| ratEGR1 | AAAATGGGTC TGGGCCCTCA GAACCCTGCC CTGTATCTTT GTACA..... |
| humanEGR1 | CTCTCAAAAG TCTATTTTTT TAA.CTGAAA ATGTAAATTT ATAAATATAT |
| | 3551 3600 |
| mouseEGR1 | TCAGGAGTTG GAGTGTTGTG GTTACCTACT GACTAGGCTG CAGTTTTTGT |
| ratEGR1 | GCATCTGTGC CATGGATTTT GTTTTCCTTG GGGTATTCTT GATGTGAAGA |
| humanEGR1 | TCAGGAGTTG GAATGTTGTA GTTACCTACT GAGTAGGCGG CGATTTTTGT |
| | 3601 3650 |
| mouseEGR1 | ATGTTATGAA CATGAAGTTC ATTATTTTGT GGTTTTATTT TACTTTGTAC |
| ratEGR1 | TAATTTGCAT ACTCTATTGT ACTATTTGGA GTTAAATTCT CACTTTGGGG |
| humanEGR1 | ATGTTATGAA CATGCAGTTC ATTATTTTGT GGTTCTATTT TACTTTGTAC |
| | 3651 3700 |
| mouseEGR1 | TTGTGTTTGC TTAAACAAAG TAACCTGTTT GGCTTATAAA CACATTGAAT |
| ratEGR1 | GAGGGGGAGC AAAGCCAAGC AAACCAATGG TGATCCTCTA TTTTGTGATG |
| humanEGR1 | TTGTGTTTGC TTAAACAAAG TGA.CTGTTT GGCTTATAAA CACATTGAAT |
| | 3701 3750 |
| mouseEGR1 | GCGCTCTATT GCCCATGG.. ..GATATGTG GTGTGTATCC TTCAGAAAAA |
| ratEGR1 | ATCCTGCTGT GACATTAGGT TTGAAACTTT TTTTTTTTTT TGAAGCAGCA |
| humanEGR1 | GCGCTTTATT GCCCATGG.. ..GATATGTG GTGTATATCC TTCCAAAAAA |
| | 3751 3800 |
| mouseEGR1 | TTAAAAGGAA AAAT...... .......... .......... .......... |
| ratEGR1 | GTCCTAGGTA TTAACTGGAG CATGTGTCAG AGTGTTGTTC CGTTAATTTT |
| humanEGR1 | TTAAAACGAA AATAAAGTAG CTGCGATTGG G ................... |
| | 3801 3850 |
| mouseEGR1 | .......... .......... .......... .......... .......... |
| ratEGR1 | GTAAATACTG CTCGACTGTA ACTCTCACAT GTGACAAAAT ACGGTTTGTT |
| humanEGR1 | .......... .......... .......... .......... .......... |
| | 3851 3900 |
| mouseEGR1 | .......... .......... .......... .......... .......... |
| ratEGR1 | TGGTTGGGTT TTTTGTTGTT TTTGAAAAAA AAATTTTTTT TTTGCCCGTC |
| humanEGR1 | .......... .......... .......... .......... .......... |
| | 3901 3950 |
| mouseEGR1 | .......... .......... .......... .......... .......... |
| ratEGR1 | CCTTTGGTTT CAAAAGTTTC ACGTCTTGGT GCCTTTGTGT GACACACCTT |
| humanEGR1 | .......... .......... .......... .......... .......... |
| | 3951 4000 |
| mouseEGR1 | .......... .......... .......... .......... .......... |
| ratEGR1 | GCCGATGGCT GGACATGTGC AATCGTGAGG GGACACGCTC ACCTCTAGCC |
| humanEGR1 | .......... .......... .......... .......... .......... |
| | 4001 4050 |
| mouseEGR1 | .......... .......... .......... .......... .......... |
| ratEGR1 | TTAAGGGGGT AGGAGTGATG TTTCAGGGGA GGCTTTAGAG CACGATGAGG |
| humanEGR1 | .......... .......... .......... .......... .......... |
| | 4051 4100 |
| mouseEGR1 | .......... .......... .......... .......... .......... |
| ratEGR1 | AAGAGGGCTG AGCTGAGCTT TGGTTCTCCA GAATGTAAGA AGAAAAATTT |
| humanEGR1 | .......... .......... .......... .......... .......... |
| | 4101 4150 |
| mouseEGR1 | .......... .......... .......... .......... .......... |
| ratEGR1 | AAAACAAAAA TCTGAACTCT CAAAAGTCTA TTTTTTTAAC TGAAAATGTA |
| humanEGR1 | .......... .......... .......... .......... .......... |
| | 4151 4200 |
| mouseEGR1 | .......... .......... .......... .......... .......... |
| ratEGR1 | GATTTATCCA TGTTCGGGAG TTGGAATGCT GCGGTTACCT ACTGAGTAGG |
| humanEGR1 | .......... .......... .......... .......... .......... |
| | 4201 4250 |
| mouseEGR1 | .......... .......... .......... .......... .......... |
| ratEGR1 | CGGTGACTTT TGTATGCTAT GAACATGAAG TTCATTATTT TGTGGTTTTA |
| humanEGR1 | .......... .......... .......... .......... .......... |
| | 4251 4300 |
| mouseEGR1 | .......... .......... .......... .......... .......... |
| ratEGR1 | TTTTACTTCG TACTTGGTT. TGCTTAAACA AAGTGACTTG TTTGGCTTAT |
| humanEGR1 | .......... .......... .......... .......... .......... |
| | 4301 4350 |
| mouseEGR1 | .......... .......... .......... .......... .......... |
| ratEGR1 | AAACACATTG AATGCGCTTT ACTGCCCATG GGATATGTGG TGTGTATCCT |
| humanEGR1 | .......... .......... .......... .......... .......... |
| | 4351 4388 |
| mouseEGR1 | .......... .......... .......... ........ |
| ratEGR1 | TCAGAAAAAT TAAAAGGAAA ATAAAGAAAC TAACTGGT |
| humanEGR1 | .......... .......... .......... ........ |

### EXPERIMENTAL DETAILS

### Example 1

Role of EGR-1 in Endothelial Cell Proliferation and Migration

Materials and Methods

Oligonucleotides and chemicals. Phosphorothioate-linked antisense oligonucleotides directed against the region comprising the translational start site of Egr-1 mRNA were synthesized commercially (Genset Pacific) and purified by high performance liquid chromatography. The target sequence of AS2 (5′-CsTsTsGsGsCsCsGsCsTsGsCsCsAsT-3′) (SEQ ID NO:16) is conserved in mouse, rat and human Egr-1 mRNA. For control purposes, we used AS2C (5′-GsCsAsCsTsTsCsTsGsCsTsGsTsCsC-3¹) (SEQ ID NO:17), a size-matched phosphorothioate-linked counterpart of AS2 with similar base composition. Phorbol-12-myristrate 13-acetate (PMA) and fibroblast growth factor-2 were purchased from Sigma-Aldrich.

Cell culture. Bovine aortic endothelial cells were obtained from Cell Applications, Inc. and used between passages 5-9. The endothelial cells were grown in Dulbecco's modified Eagles' medium (Life Technologies), pH 7.4, containing 10% fetal bovine serum supplemented with 50 μg/mL streptomycin and 50 IU/mL penicillin. The cells were routinely passaged with trypsin/EDTA and maintained at 37° C. in a humidified atmosphere of 5% CO₂/95% air.

Transient transfection analysis and, CAT assay. The endothelial cells were grown to 60-70% confluence in 100 mm. dishes and transiently transfected with 10 μg of the indicated chloramphenicol acetyl transferase (CAT)-based promoter reporter construct using FuGENE6 (Roche). The cells were rendered growth-quiescent by incubation 48 h in 0.25% FBS, and stimulated with various agonists for 24 h prior to harvest and assessment of CAT activity. CAT activity was measured and normalized to the concentration of protein in the lysates (determined by Biorad Protein Assay) as previously described (Khachigian, L. M. and Chesterman, C. N. (1999), Circ. Res., 84:1258-1267).

Northern blot analysis. Total RNA (12 μg/well) of growth-arrested endothelial cells (prepared using TRIzol Reagent (Life Technologies) in accordance with the manufacturer's instructions) previously exposed to various agonists for 1 h was resolved by electrophoresis on denaturing 1% agarose-formialdehyde gels. Following transfer overnight to Hybond-N+ nylon membranes (Amersham), the blots were hybridized with ³²P-labeled Egr-1 cDNA prepared using the Nick Translation Kit overnight (Roche). The membranes were washed and radioactivity visualized by autoradiography as previously described (Khachigian et al., 1995).

RT-PCR. Reverse transcription was performed with 8 μg of total RNA using M-MLV reverse transcriptase. Egr-1 cDNA was amplified (334 bp product (Delbridge, G. J. and Khachigian, L. M. (1997), Circ. Res., 81:282-288)) using Taq polymerase by heating for 1 min at 94° C., and cycling through 94° C. for 1 min, 94° C. for 1 min, 55° C. for 1 min, and 72° C. for 1 min. Following thirty cycles, a 5 min extension at 72° C. was carried out. Samples were electrophoresed on 1.5% agarose gel containing ethidium bromide and photographed under ultraviolet illumination. β-actin amplification (690 bp product) was performed essentially as above. The sequences of the primers were: Egr-1 forward primer (5′-GCA CCC AAC AGT GGC AAC-3′) (SEQ ID NO:18), Egr-1 reverse primer (5′-GGG ATC ATG GGA ACC TGG-3′) (SEQ ID NO: 19), β-actin forward primer (5′-TGA CGG GGT CAC CCA CAC TGT GCC CAT CTA 3′) (SEQ ID NO:20), and β-actin reverse primer (5′-CTA GAA GCA TTT GCG GTG GAC GAT GGA GGG-3′) (SEQ ID NO:21).

Antisense oligonucleotide delivery and Western blot analysis. Growth arrested cells in 100 mm dishes were incubated with the indicated oligonucleotides 24 h and 48 h after the initial change of medium. When oligonucleotide was added a second time, the cells were incubated with various concentrations of insulin and harvest 1 h subsequently. The cells were washed in cold phosphate-buffered saline (PBS), pH 7.4, and solubilized in RIPA buffer (150 mM NaCl, 50 mM Tris-HCI, pH 7.5, 1% sodium deoxycholate, 0.1% SDS, 1% Triton X-100, 5 mM EDTA, 1% trasylol, 10 μg/ml leupeptin, 1% aprotinin, 2 μM PMSF). Lysates were resolved by electrophoresis on 8% denaturing SDS-polyacrylamide gels, transferred to PDVF nylon membranes (NEN-DuPont), blocked with skim milk powder, then incubated with polyclonal antibodies to Egr-1 (Santa Cruz Biotechnology, Inc) and monoclonal horseradish peroxidase-linked mouse anti-rabbit Ig secondary antibodies followed by chemiluminescent detection (NEN-DuPont).

³H-Thymidine incorporation into DNA. Growth-arrested endothelial cells at 90% confluence in 96 well plates were incubated twice with the oligonucleotides prior to the addition of insulin. When signaling inhibitors (PD98059, SB202190, wortmannin) were used in experiments, these agents were added 2 h before the addition of insulin. After 18 h of exposure to insulin, the cells were pulsed with 200,000 cpm/well of methyl-3H thymidine (NEN-DuPont) for 6 h. Lysates were prepared by washing first in cold PBS, pH 7.4, then fixing with cold 10% trichloroacetic acid, washing with cold ethanol and solubilizing in 0.1 M NaOH. ³H-Thymidine in the lysates was quantitated with ACSII scintillant using β-scintillation counter (Packard).

In vitro injury. Growth-arrested cells at 90% confluence were incubated with antisense oligonucleotides and insulin at various concentrations as described above, then were scraped by drawing a sterile wooden toothpick across the monolayer (Khachigian et al., 1996). Following 48-72 h, the cells were fixed in 4% formalin, stained with hemotoxylin/eosin then photographed.

HMEC-1 culture and proliferation assay. SV40-transformed HMEC-1 cells were grown in MCDB 131 medium with EGF (10 ng/ml) and hydrocortisone (1 μg/ml) supplements and 10% FBS. Forty-eight h after incubation in serum-free medium without supplements, the cells were transfected with the indicted DNA enzyme (0.4 μM) and transfected again 72 h after the change of medium, when 10% serum was added. The cells were quantitated by Coulter counter, 24 h after the addition of serum.

Antisense Egr-1 mRNA overexpression. Bovine aortic endothelial cells or rat vascular smooth muscle cells were grown to 60% confluence in 96-well plates then transfected with 3 μg of construct pcDNA3-a/SEgr-1 (in which a 137Bp fragment of Egr-1 cDNA (732-869) was cloned in antisense orientation into the BamHI/EcoRI site of pcDNA3), or pcDNA3 alone, using Fugene6 in accordance with the manufacturer's instructions. Growth arrested cells were incubated with 5% FBS in Waymouth's medium (SMC) or DMEM (EC) and trypisinised after 3 days prior to quantitation of the cell populations by Coulter counting.

### Results and Discussion

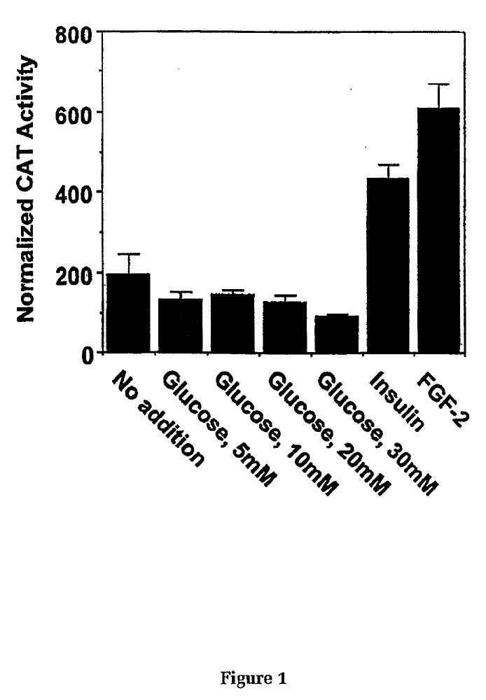

Insulin, but not Glucose, Stimulates Egr-1 Activity in Vascular Endothelial Cells. High glucose may activate normally-quiescent vascular endothelium by stimulating mitogen-activated protein (MAP) kinase activity and the expression of immediate-early genes (Frodin, M. et al. (1995), J. Biol. Chem., 270:7882-7889 and Kang, M. J. (1999), Kidney Int., 55:2203-2214). These signaling and transcriptional events may, in turn, induce the expression of other genes whose products then alter endothelial phenotype and facilitate the development of lesions. To determine the effect of glucose on Egr-1 activity in vascular endothelial cells, we performed transient transfection analysis in endothelial cells transfected with pEBS1³foscat, a chloramphenical acetyltransferase (CAT)-based reporter vector driven by three high-affinity Egr-1 binding sites placed upstream of the c-fos TATA box (Gashler, A. L. et al. (1993), Mol. Cell. Biol., 13:4556-4571). Exposure of growth-arrested endothelial cells to various concentrations of glucose (5 to 30 mM) over 24 h did not increase Egr-1 binding activity (FIG. 1). However, Egr-1 binding activity did increase in cells exposed to insulin (100 nM) (FIG. 1). Reporter activity also increased upon incubation with FGF-2, a known inducer of Egr-1 transcription and binding activity in vascular endothelial cells (Santiago, F. S. et al. (1999), Am. J. Pathol., 154:937-944) (FIG. 1).

Insulin and FGF-2 Induce Egr-1 mRNA Expression in Vascular Endothelial Cells. The preceding findings using reporter gene analysis provided evidence for increased Egr-1 expression in endothelial cells exposed to insulin. We next used reverse transcription-polymerase chain reaction (RT-PCR) and Northern blot analysis to demonstrate directly the capacity of insulin to increase levels of Egr-1 mRNA. RT-PCR revealed that Egr-1 is weakly expressed in growth-quiescent endothelial cells (data not shown). Insulin, like FGF-2, increased Egr-1 expression within 1 h of exposure to the agonist. In contrast, levels of β-actin mRNA were unchanged. Northern blot analysis confirmed these qualitative data by demonstrating that insulin, FGF-2, and phorbol 12-myristate 13-acetate (PMA), a second potent inducer of Egr-1 expression (Khachigian et al., 1995) elevated steady-state Egr-1 mRNA levels within 1 h without increasing levels of ribosomal 28S and 18S mRNA (data not shown).

Insulin-Stimulated Egr-1 Protein Synthesis in Endothelial Cells is Inhibited by Antisense Oligonucleotides Targeting Egr-1 mRNA. To reconcile our demonstration of insulin-induced Egr-1 mRNA expression with the binding activity of the transcription factor (FIG. 1), we performed Western immunoblot analysis using polyclonal antibodies directed against Egr-1 protein. Insulin (at 100 nM and 500 nM) induced Egr-1 protein synthesis in growth-arrested endothelial cells within 1 h (data not shown). These findings, taken together, demonstrate that insulin elevates Egr-1 mRNA, protein and binding activity in vascular endothelial cells.

We recently developed phosphorothioate-based antisense oligonucleotides targeting the translational start site in Egr-1 mRNA (Santiago, F. S. et al. (1999), Am. J. Pathol., 155:897-905). These oligonucleotides lack phosphorothioate G-quartet sequences that have been associated with non-specific biological activity (Stein, C.A. (1997), Ciba Foundation Symposium, 209:79-89). Western blot analysis revealed that prior incubation of growth-arrested endothelial cells with 0.8 μM antisense Egr-1 oligonucleotides (AS2) inhibited insulin-inducible Egr-1 protein synthesis, despite equal loading of protein. The lack of attenuation in insulin-inducible Egr-1 protein following exposure of the cells to an identical concentration of AS2C demonstrates the sequence-specific inhibitory effect of the antisense Egr-1 oligonucleotides.

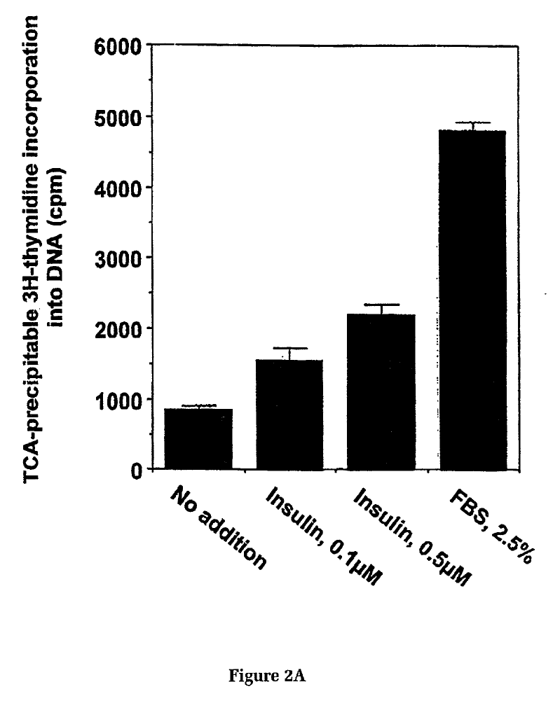

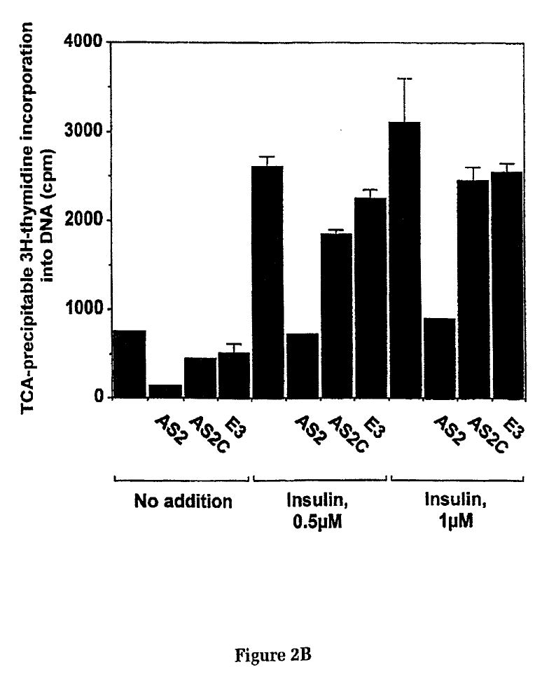

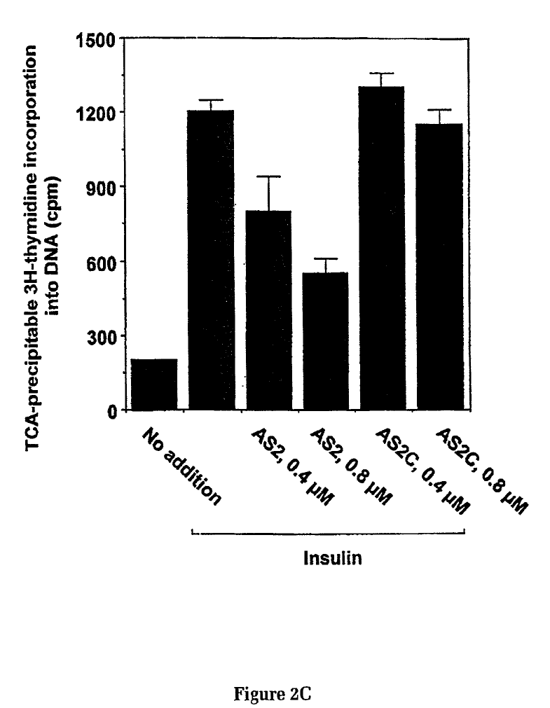

Insulin Stimulates Endothelial Cell DNA Synthesis which is Inhibited by Antisense Oligonucleotides Targeting Egr-1 mRNA. These oligonucleotides, which attenuate the induction of Egr-1 protein, were used in ³H-thymidine incorporation assays to determine the involvement of Egr-1 in insulin inducible DNA synthesis. This assay evaluates ³H-thymidine uptake into DNA precipitable with trichloroacetic acetic (TCA) (Khachigian, L. M and Chesterman, C. N. (1992), J. Biol. Chem., 267:7478-7482). In initial experiments, growth-arrested endothelial cells exposed to insulin (100 nM) increased the extent of DNA synthesis by 100%, whereas 500 nM insulin caused a 200% increase in DNA synthesis (FIG. 2A).

We next determined the effect of AS2 and AS2C on insulin-inducible endothelial DNA synthesis. In the absence of added insulin, AS2 (0.8 μM) inhibited basal endothelial DNA synthesis facilitated by low concentrations of serum (0.25% v:v) (FIG. 2B). In contrast, the scrambled control (0.8 μM) or a third oligonucleotide, E3 (0.8 AM), a size-matched phosphorothioate directed toward another region of Egr-1 mRNA (Santiago et al., 1999) had little effect on basal DNA synthesis (FIG. 2B). Furthermore, unlike AS2 and E3, AS2 significantly inhibited DNA synthesis inducible by insulin (500 nM and 1000 nM) (FIG. 2B). To demonstrate concentration-dependent inhibition of DNA synthesis, we incubated the endothelial cells with 0.4 μM as well as 0.8 μM of Egr-1 oligonucleotide. Since this lower concentration of AS2 inhibited ³H-thyrnidine incorporation less effectively (compare to AS2C) indicates dose-dependent and sequence-specific inhibition by the antisense Egr-1 oligonucleotide (FIG. 2C). These findings thus demonstrate the requirement for Egr-1 protein in endothelial cell DNA synthesis inducible by insulin.

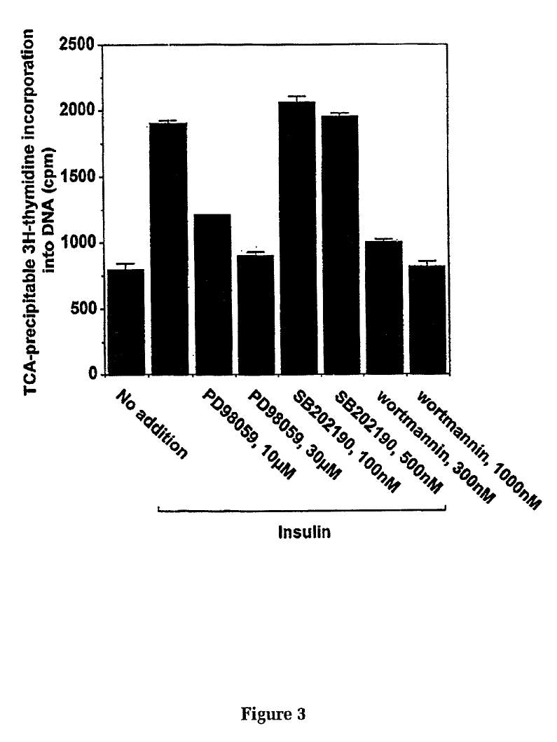

Insulin-Stimulated DNA Synthesis in Endothelial Cells is Inhibited by PD98059 and Wortmannin, But Not by SB202190. Inducible Egr-1 transcription is governed by the activity of extracellular signal-regulated kinase (ERK) (Santiago et al., 1999) which phosphorylates factors at serum response elements in the Egr-1 promoter (Gashler et al., 1995). Since there is little known about signaling pathways mediating insulin-inducible proliferation of vascular endothelial cells, we determined the relevance of MEK/ERK in this process using the specific MEK/ERK inhibitor, PD98059. This compound (at 10 and 30 μM) inhibited insulin-inducible DNA synthesis in a dose-dependent manner (FIG. 3). Likewise, wortmannin (0.3 and 1 μM), the phosphatidylinositol 3-kinase inhibitor which also inhibits c-Jun N-terminal kinase (JNK) (Ishizuka, T. et al., (1999), J. Immunol., 162:2087-2094; Day, F. L. et al., (1999), J. Biol. Chem, 274:23726-23733; Kumahara, E. et al., (1999), J. Biochem., 125:541-553), ERK (Barry, O. P. et al., (1999), J. Biol. Chem., 274:7545-7556) and p38 kinase (Barry et al., 1999) inhibited DNA synthesis in a dose-dependent manner (FIG. 3). In contrast, SB202190 (100 and 500 nM), a specific p38 kinase inhibitor failed to affect DNA synthesis (FIG. 3). These findings demonstrate the critical role for MEK/ERK, and possibly JNK, in insulin-inducible endothelial cell proliferation, and the lack of p38 kinase involvement in this process.

Insulin Stimulates Endothelial Cell Regrowth After Mechanical Injury In Vitro in an Egr-1, Dependent Manner. Mechanically wounding vascular endothelial (and smooth muscle) cells in culture results in migration and proliferation at the wound edge and the eventual recoverage of the denuded area. We hypothesized that insulin would accelerate this cellular response to mechanical injury. Acutely scraping the growth-quiescent (rendered by 48 h incubation in 0.25% serum) endothelial monolayer resulted in a distinct wound edge (data not shown). Continued incubation of the cultures in medium containing low serum for a further 3 days resulted in weak regrowth in the denuded zone but aggressive regrowth in the presence of optimal amounts of serum (10%). When insulin (500 nM) was added to growth-quiescent cultures at the time of injury the population of cells in the denuded zone significantly increased, albeit as expected, less efficiently than the 10% serum control.

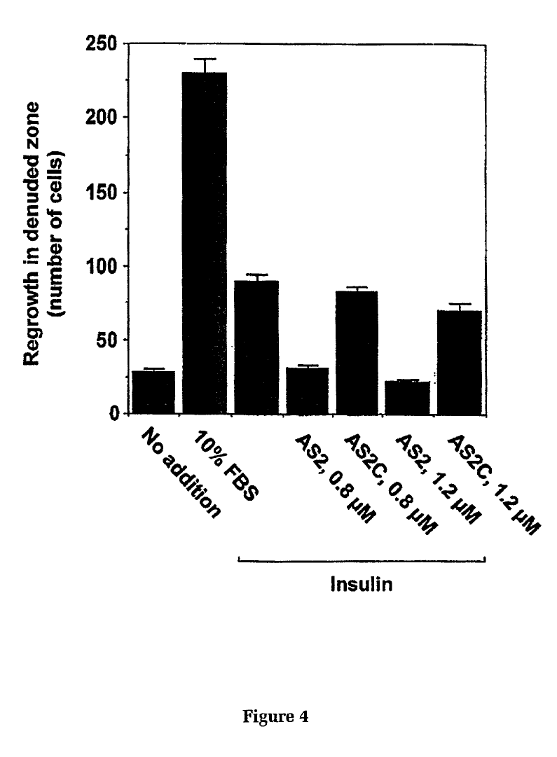

To investigate the involvement of Egr-1 in endothelial regrowth potentiated by insulin after injury we incubated the cultures with antisense Egr-1 oligonucleotides prior to scraping and again at the time of injury and the addition of insulin. AS2 (0.8 μM) significantly inhibited endothelial regrowth stimulated by insulin. In contrast, regrowth in the presence of AS2C (0.8 μM) was not significantly different from cultures in which oligonucleotide was omitted. Similar findings were observed when higher concentrations (1.2 μm) of AS2 and AS2C were used. Thus, endothelial regrowth after injury stimulated by insulin proceeds in an Egr-1-dependent manner. These observations are quantitated in FIG. 4.

These results show that insulin-induced proliferation and regrowth after injury are processes critically dependent upon the activation of Egr-1 Northern blot, RT-PCR and Western immunoblot analysis reveal that insulin induces Egr-1 mRNA and protein expression. Antisense oligonucleotides which block insulin-induced synthesis of Egr-1 protein in a sequence-specific and dose-dependent manner, also inhibit proliferation and regrowth after mechanical injury. These findings using nucleic acids specifically targeting Egr-1 demonstrate the functional involvement of this transcription factor in endothelial growth.

Insulin signaling involves the activation of a growing number of immediate-early genes and transcription factors. These include c-fos (Mohn, K. L. et al., (1990), J. Biol. Chem., 265:21914-21921; Jhun, B. H. et al., (1995), Biochemistry, 34:7996-8004; Harada, S. et al., (1996), J. Biol. Chem., 271:30222-30226), c-jun (Mohn et al., 1990), nuclear factor-KB (Bertrand, F. et al., (1998), J. Biol. Chem., 273:2931-2938, SOCS3 (Emanuelli, B. et al., (2000), J. Biol. Chem., 275:15985-15991) and the forkhead transcription factor FKHR (Nakae, J. et al., (1999), J. Biol. Chem., 274:15982-15985). Insulin also induces the expression of Egr-1 in mesangial cells (Solow, B. T. et al., (1999) Arch. Biochem. Biophys., 370:308-313), fibroblasts (Jhun et al., 1995), adipocytes (Alexander-Bridges, M. et al., (1992), Mol. Cell. Biochem., 109:99-105) and Chinese hamster ovary cells (Harada et al., 1996). This study is the first to describe the induction of Egr-1 by insulin in vascular endothelial cells.

Insulin activates several subclasses within the MAP kinase superfamily, including ERK, JNK and p38 kinase (Guo, J. H. et al., (1998), J. Biol, Chem., 273:16487-16493). Our findings indicate that the specific ERK inhibitor PD98059, which binds to MEK and prevents phosphorylation by Raf, inhibits insulin-inducible endothelial cell proliferation. Egr-1 transcription is itself dependent upon the phosphorylation activity of ERK via its activation of ternary complex factors (such as Elk-1) at serum response elements (SRE) in the Egr-1 promoter. Six SREs appear in the Egr-1 promoter whereas only one is present in the c-fos promoter (Gashler et al., 1995). PD98059 blocks insulin-inducible Elk-1 transcriptional activity at the c-fos SRE in vascular cells (Xi, X. P. et al., (1997), FEBS Lett., 417:283-286. These published findings are consistent with the present demonstration of the involvement of Egr-1 in insulin-inducible proliferation.

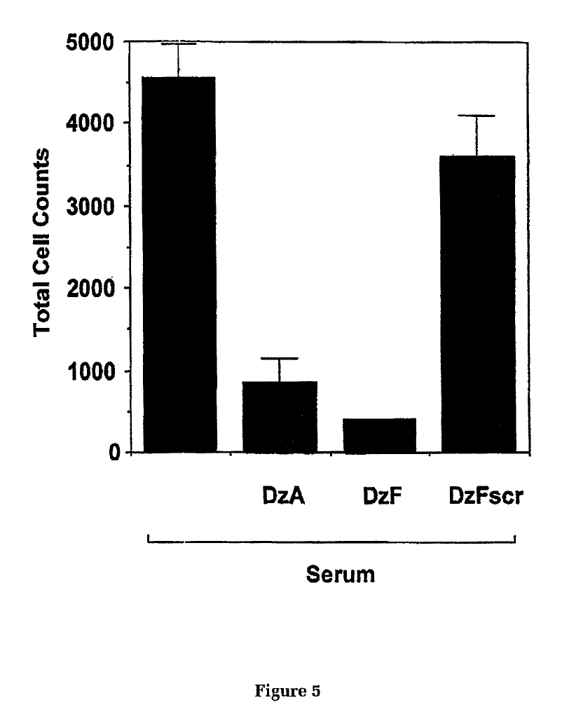

To provide evidence, independent of insulin, that endothelial proliferation is an Egr-1 dependent process, we incubated human microvascular endothelial cells (HMEC-1) separately with two DNA enzymes (DzA and DzF) each targeting different sites in human EGR-1 mRNA, at a final concentration of 0.4 μM. DzA and DzF both inhibited HMEC-1 replication (total cell counts) in the presence of 5% serum (FIG. 5). In contrast, DzFscr, was unable to modulate proliferation at the same concentration (FIG. 5). DzFscr bears the same active 15 nt catalytic domain as DzF and has the same net charge but has scrambled hybridizing arms. These data obtained using a second endothelial cell type demonstrate inhibition of endothelial proliferation using sequence-specific strategies targeting human EGR-1.

Finally, we found that CMW-mediated overexpression of antisense Egr-1 mRNA inhibited proliferation of both endothelial cells and smooth muscle cells. Replication of both endothelial and smooth muscle cell pcDNA3-A/SEgr-1 transfectants was significantly lower than those transfected with the backbone vector alone, pcDNA3 (data not shown). These findings demonstrate that antisense EGR mRNA strategies can inhibit proliferation of arterial endothelial cells and at least one other vascular cell type.

Despite the availability and clinical use of a large number of chemotherapeutic agents for the clinical management of neoplasia, solid tumours remain a major cause of mortality in the Western world. Drugs currently used to treat such tumours are generally non-specific poisons that can be toxic to non-cancerous tissue and require high doses for efficacy. There is growing evidence that the cellular and molecular mechanisms underlying tumour growth involves more than just tumour cell proliferation and migration. Importantly, tumour growth and metastasis are critically dependent upon ongoing angiogenesis, the process new blood vessel formation (Crystal et al., 1999). The present findings, which demonstrate that Egr-1 is critical in vascular endothelial cell replication and migration, strongly implicate this transcription factor as a key regulator in angiogenesis and tumorigenesis.

### Example 2

Characterisation of DNAzyme Targeting Rat Egr-1 (NGFI-Al)

Materials and Methods

ODN synthesis. DNAzymes were synthesized commercially (Oligos Etc., Inc.) with an inverted T at the 3′ position unless otherwise indicated. Substrates in cleavage reactions were synthesized with no such modification. Where indicated ODNs were 5′-end labeled with γ³²P-DATP and T4 polynucleotide kinase (New England Biolabs). Unincorporated label was separated from radiolabeled species by centrifugation on Chromaspin-lo columns (Clontech).

In vitro transcript and cleavage experiments. A ³²P labelled 206 nt NGFI-A RNA transcript was prepared by in vitro transcription (T3 polyinerase) of plasmid construct pJDM8 (as described in Milbrandt, J. A., (1987), Science, 238:797-799), the entire contents of which are incorporated herein by reference) previously cut with Bgl II Reactions were performed in a total volume of 20 μl containing 10 mM MgCl₂, 5 mM Tris pH 7.5, 150 mM NaCl₂ 4.8 pmol of in vitro transcribed or synthetic RNA substrate and 60 pmol DNAzyme (1:12.5 substrate to DNAzyme ratio), unless otherwise indicated. Reactions were allowed to proceed at 37° C. for the times indicated and quenched by transferring an aliquot to tubes containing formamide loading buffer (Sambrook, J. et al., (1989), Molecular clonning: a laboratory manual, Cold Spring Harbor Laboratory Press, Plainview, N.Y.). Samples were run on 12% denaturing polyacrylamide gels and autoradiographed overnight at −80° C.

Culture conditions and DNAzyme transfection. Primary rat aortic SMCs were obtained from Cell Applications, Inc., and grown in Waymouth's medium, pH 7.4, containing 10% fetal bovine serum (FBS), 50 μg/ml streptomycin and 50 IU/ml penicillin at 37° C. in a humidified atmosphere of 5% CO₂ SMCs were used in experiments between passages 3-7. Pup rat SMCs (WKY12-22 (as described in Lernire et al, 1994, the entire contents of which are incorporated herein by reference)) were grown under similar conditions. Subconfluent (60-70%) SMCs were incubated in serum-free medium (SFM) for 6 h prior to DNAzyme (or antisense ODN, where indicated) transfection (0.1 μM) using Superfect in accordance with manufacturer's instructions (Qiagen). After 18 h, the cells were washed with phosphate-buffered saline (PBS), pH 7.4 prior to transfection a second time in 5% FBS.

Northern blot analysis. Total RNA was isolated using the TRIzol reagent (Life Technologies) and 25 μg was resolved by electrophoresis prior to transfer to Hybond-N+ membranes (NEN-DuPont). Prehybridization, hybridization with (α³²P-dCTP-labeled Egr-1 or β-Actin cDNA, and washing was performed essentially as previously described (Khachigian et al., 1995).

Western blot analysis. Growth-quiescent SMCs in 100 mm plates (Nunc-InterMed) were transfected with ED5 or ED5SCR as above, and incubated with 5% FBS for 1 h. The cells were washed in cold PBS, pH 7.4, and extracted in 150 mM NaCl, 50 mM Tris-HCI, pH 7.5, 10% sodium deoxycholate, 0.1% SDS, 1% Triton X-100, 5 mM EDTA, 1% trasylol, 10μ/ml leupeptin, 1% aprotinin and 2 mM PMSF. Twenty four μg protein samples were loaded onto 10% denaturing SDS-polyacrylamide gels and electroblotted onto PVDF nylon membranes (NEN-DuPont). Membranes were air dried prior to blocking with non-fat skim milk powder in PBS containing 0.05% (w:v) Tween 20. Membranes were incubated with rabbit antibodies to Egr-1 or Sp1 (Santa Cruz Biotechnology, Inc.) (1:1000) then with HRP-linked mouse anti-rabbit Ig secondary antiserum (1:2000). Where mouse monoclonal c-Fos (Santa Cruz Biotechnology, Inc.) was used, detection was achieved with HRP-linked rabbit anti-mouse Ig. Proteins were visualized by chemiluminescent detection (NEN-DuPont).

Assays of cell proliferation. Growth-quiescent SMCs in 96-well titer plates (Nunc-InterMed) were transfected with ED5 or ED5SCR as above, then exposed to 5% FBS at 37° C. for 72 h. The cells were rinsed with PBS, pH 7.4, trypsinized and the suspension was quantitated using an automated Coulter counter.

Assessment of DNAzyme stability. DNAzymes were 5′-end labeled with γ³²P-DATP and separated from free label by centrifugation. Radiolabeled DNAzymes were incubated in 5% FBS or serum-free medium at 37° C. for the times indicated. Aliquots of the reaction were quenched by transfer to tubes containing formamide loading buffer (Sambrook et al., 1989). Samples were applied to 12% denaturing polyacrylamide gels and autoradiographed overnight at −80° C.

SMC wounding assay. Confluent growth-quiescent SMCs in chamber slides (Nunc-InterMed) were exposed to ED5 or ED5SCR for 18 h prior to a single scrape with a sterile toothpick. Cells were treated with mitomycin C (Sigma) (20 μM) for 2 h prior to injury (Pitsch et al, 1996; Horodyski and Powell, 1996). Seventy-two h after injury, the cells were washed with PBS, pH 7.4, fixed with formaldehyde then stained with hematoxylin-eosin.

Rat arterial ligation model and analysis. Adult male Sprague Dawley rats weighing 300-350 g were anaesthetised using ketamine (60 mg/kg, i.p.) and xylazine (8 mg/kg, i.p.). The right common carotid artery was exposed up to the carotid bifurcation via a midline neck incision. Size 6/0 non absorbable suture was tied around the common carotid proximal to the bifurcation, ensuring cessation of blood flow distally. A 200 μl solution at 4° C. containing 500 μg of DNAzyme (in DEPC-treated H₂O), 1 mM MgCl₂, 30 μl of transfecting agent (Fugene 6) and Pluronic gel P127 (BASF) was applied around the vessel in each group of 5 rats, extending proximally from the ligature for 12-15 mm. These agents did not inhibit the solidification of the gel at 37° C. After 3 days, vehicle with or without 500 μg of DNAzyme was administered a second time. Animals were sacrificed 18 days after ligation by lethal injection of phenobarbitone, and perfusion fixed using 10% (v:v) formaldehyde perfused at 120 mm Hg. Both carotids were then dissected free and placed in 10% formaldehyde, cut in 2 mm lengths and embedded in 3% (w:v) agarose prior to fixation in paraffin. Five μm sections were prepared at 250 μm intervals along the vessel from the point of ligation and stained with hematoxylin and eosin. The neointimal and medial areas of 5 consecutive sections per rat were determined digitally using a customized software package (Magellan) (Halasz, S. and Martin, P., (1984), Proc. Royal Microscop. Soc., 19:312) and expressed as a mean ratio per group of 5 rats.

Results and Discussion

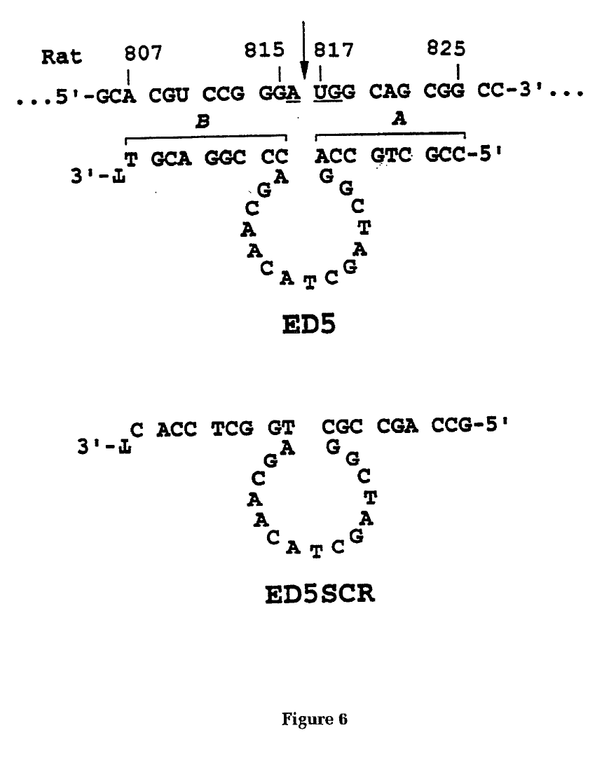

The 7×7 nt arms flanking the 15 nt DNAzyme catalytic domain in the original DNAzyme design (Santoro, S. W. and Joyce, G. F. (1997), Natl. Acad. Sci. USA, 94:4262-4266) were extended by 2 nts per arm for improved specificity (L.-Q. Sun, data not shown) (FIG. 6). The 3′terminus of the molecule was capped with an inverted 3′-3′-linked thymidine (T) to confer resistance to 3′->5′exonuclease digestion. The sequence in both arms of ED5 was scrambled (SCR) without altering the catalytic domain to produce DNAzyme ED5SCR (FIG. 6).

A synthetic RNA substrate comprised of 23 nts, matching nts 805 to 827 of NGFI-A mRNA (FIG. 6) was used to determine whether ED5 had the capacity to cleave target RNA. ED5 cleaved the ³²P-5′-end labeled 23-mer within 10 min (data not shown). The 12-mer product corresponds to the length between the A(816)-U(817) junction and the 5′end of the substrate (FIG. 6). In contrast, ED5SCR had no demonstrable effect on this synthetic substrate. Specific EDS catalysis was further demonstrated by the inability of the human equivalent of this DNAzyme (hED5) to cleave the rat substrate over a wide range of stoichiometric ratios (data not shown). Similar results were obtained using ED5SCR (data not shown). hED5 differs from the rat ED5 sequence by 3 of 18 nts in its hybridizing arms (Table 2). The catalytic effect of ED5 on a ³²P-labeled 206 nt fragment of native NGFI-A mRNA prepared by in vitro transcription was then determined. The cleavage reaction produced two radiolabeled species of 163 and 43 nt length consistent with DNAzyme cleavage at the A(816)-U(817) junction. In other experiments, ED5 also cleaved a ³²P-labeled NGFI-A transcript of 1960 nt length in a specific and time-dependent manner (data not shown).

### Table 2. DNAzyme Target Sites in mRNA.

Similarity between the 18 nt arms of ED5 or hED5 and the mRNA of rat NGFI-A or human EGR-1 (among other transcription factors) is expressed as a percentage. The target sequence of ED5 in NGFI-A mRNA is 5′ A CGU CCG GGA UGG CAG CGG 31 (SEQ ID NO:22) (rat NGFI-A sequence), and that of hED5 in EGR-1 is 5′ U CGU CCA GGA UGG CCG CGG 31 (SEQ ID NO:23) (Human EGR-1 sequence). Nucleotides in bold indicate mismatches between rat and human sequences. Data obtained by a gap best fit search in ANGIS using sequences derived from Genbank and EMBL. Rat sequences for Sp1 and c-Fos have not been reported.

| | Accession | Best homology over 18 nts | |
|---|---|---|---|
| Gene | number | (%) | |
| | | ED5 | hED5 |
| Rat NGFI-A | M18416 | 100 | 84.2 |
| Human EGR-1 | X52541 | 84.2 | 100 |
| Murine Spl | AF022363 | 66.7 | 66.7 |
| Human c-Fos | K00650 | 66.7 | 66.7 |
| Murine c-Fos | X06769 | 61.1 | 66.7 |
| Human Spi | AF044026 | 38.9 | 28.9 |

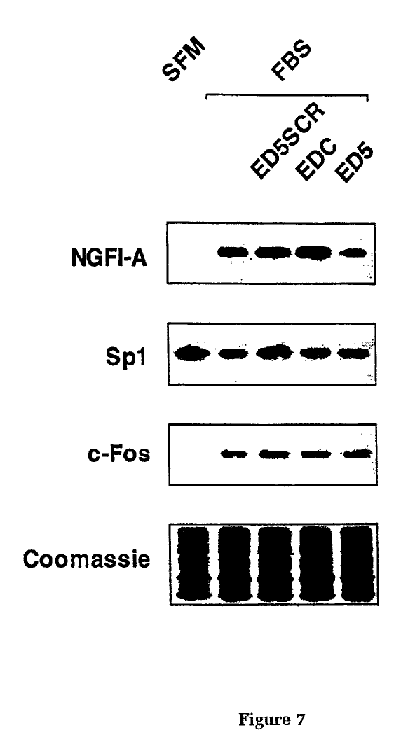

To determine the effect of the DNAzymes on endogenous levels of NGFI-A mRNA, growth-quiescent SMCs were exposed to ED5 prior to stimulation with serum. Northern blot and densitometric analysis revealed that ED5 (0.1 μM) inhibited serum-inducible steady-state NGFI-A mRNA levels by 55% (data not shown), whereas ED5SCR had no effect (data not shown). The capacity of ED5 to inhibit NGFI-A synthesis at the level of protein was assessed by Western blot analysis. Serum-induction of NGFI-A protein was suppressed by ED5. In contrast, neither ED5SCR nor EDC, a DNAzyme bearing an identical catalytic domain as ED5 and ED5SCR but flanked by nonsense arms had any influence on the induction of NGFI-A (FIG. 7). ED5 failed to affect levels of the constitutively expressed, structurally-related zinc-finger protein, Sp1 (FIG. 7). It was also unable to block serum-induction of the immediate-early gene product, c-Fos (FIG. 7) whose induction, like NGFI-A, is dependent upon serum response elements in its promoter and phosphorylation mediated by extracellular-signal regulated kinase (Treisman, R. (1990), Curr. Opin. Genet. Develop., 1:47-58; Treisman, R. (1994), Curr. Opin. Genet. Develop., 4:96-101; Treisman, R. (1995) EMBO J., 14:4905-4913; and Gashler and Sukhatme, 1995). These findings, taken together, demonstrate the capacity of ED5 to inhibit production of NGFI-A mRNA and protein in a gene-specific and sequence-specific manner, consistent with the lack of significant homology between its target site in NGFI-A mRNA and other mRNA (Table 2).

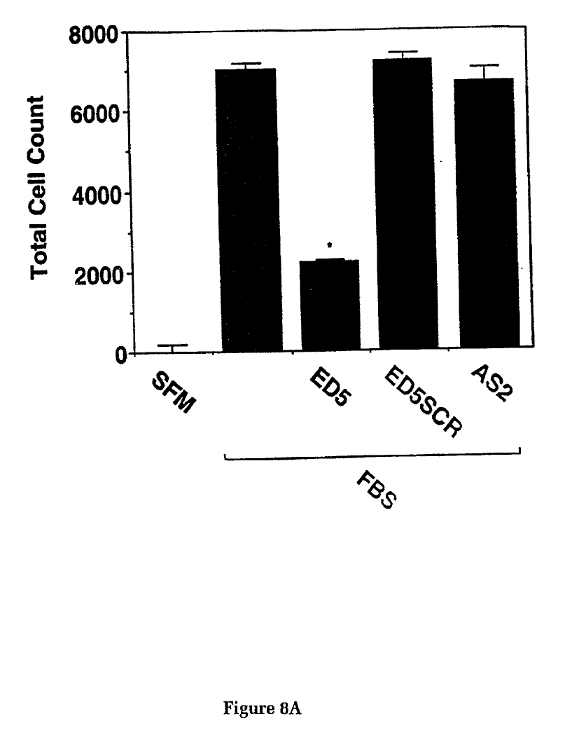

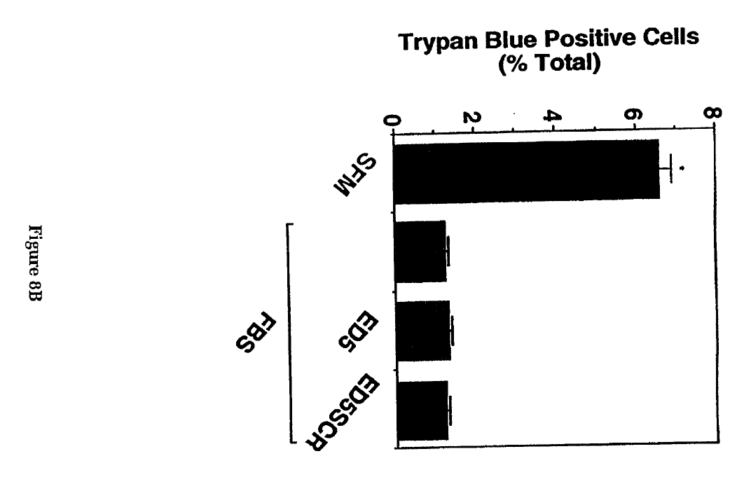

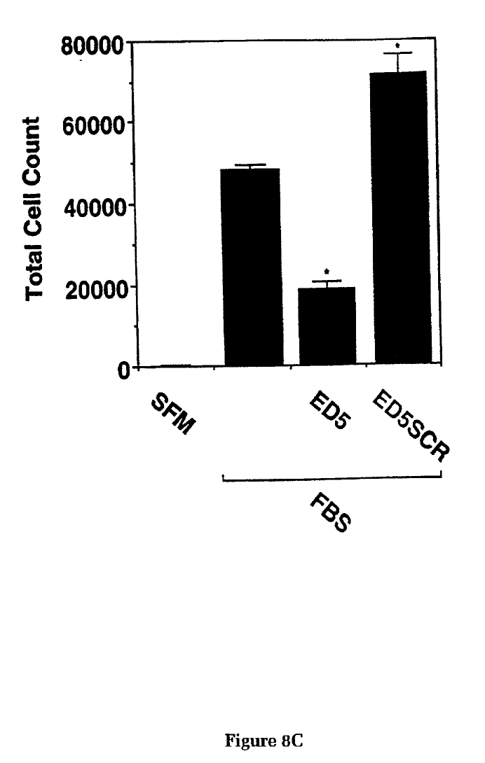

The effect of ED5 on SMC replication was next determined. Growth quiescent SMCs were incubated with DNAzyme prior to exposure to serum and the assessment of cell numbers after 3 days. ED5 (0.1 μM) inhibited SMC proliferation stimulated by serum by 70% (FIG. 8a). In contrast, ED5SCR failed to influence SMC growth (FIG. 8a). AS2, an antisense NGFI-A ODN able to inhibit SMC growth at 1 μM failed to inhibit proliferation at the lower concentration (FIG. 8a). Additional experiments revealed that ED5 also blocked serum-inducible ³H-thymidine incorporation into DNA (data not shown). ED5 inhibition was not a consequence of cell death since no change in morphology was observed, and the proportion of cells incorporating Trypan Blue in the presence of serum was not influenced by either DNAzyme (FIG. 8b).

Cultured SMCs derived from the aortae of 2 week-old rats (WKY12-22) are morphologically and phenotypically similar to SMCs derived from the neointima of balloon-injured rat arteries (Seifert, R. A. et al., (1984), Nature, 311:669-671) and Majesky, M. W. et al., (1992), Circ. Res., 71:759-768). The epitheloid appearance of both WKY12-22 cells and neointimal cells contrasts with the elongated, bipolar nature of SMCs derived from normal quiescent media (Majesky, M. W. et al., (1988), Proc. Natl. Acad. Sci. USA, 85:1524-1528). WKY12-22 cells grow more rapidly than medial SMCs and overexpress a large number of growth regulatory molecules (Lemire, J. M. et al., (1994), Am. J. Pathol., 144:1068-1081), such as NGFI-A (Rafty, L. A. and Khachigian, L. M. (1998), J. Biol. Chem., 273:5758-5764), consistent with a “synthetic” phenotype (Majesky et al., 1992; Campbell, G. R. and Campbell, J. H., (1985), Exp. Mol. Pathol., 42:139-162). ED5 attenuated serum-inducible WKY12 22 proliferation by approximately 75% (FIG. 8c). ED5SCR had no inhibitory effect; surprisingly, it appeared to stimulate growth (FIG. 8c).

Trypan Blue exclusion revealed that DNAzyme inhibition was not a consequence of cytotoxicity (data not shown).

To ensure that differences in the biological effects of ED5 and ED5SCR were not the consequence of dissimilar intracellular localization, both DNAzymes were 5′-end labeled with fluorescein isothiocyanate (FITC) and incubated with SMCs. Fluorescence microscopy revealed that both FITC-ED5 and FITC-ED5SCR localized mainly within the nuclei. Punctate fluorescence in this cellular compartment was independent of DNAzyme sequence. Fluorescence was also observed in the cytoplasm, albeit with less intensity. Cultures not exposed to DNAzyme showed no evidence of autofluorescence.

Both molecules were 5′-end labeled with γ³²P-DATP and incubated in culture medium to ascertain whether cellular responsiveness to ED5 and ED5SCR was a consequence of differences in DNAzyme stability. Both ³²P-ED5 and ³²P-ED5SCR remained intact even after 48 h (data not shown). In contrast to ³²P-ED5 bearing the 3′ inverted T, degradation of P-ED5 bearing its 3′T in the correct orientation was observed as early as 1 h. Exposure to serum-free medium did not result in degradation of the molecule even after 48 h (data not shown). These findings indicate that inverse orientation of the 3′ base in the DNAzyme protects the molecule from nucleolytic cleavage by components in serum.

Physical trauma imparted to SMCs in culture results in outward migration from the wound edge and proliferation in the denuded zone. We determined whether ED5 could modulate this response to injury by exposing growth-quiescent SMCs to either DNazyme and Mitomycin C, an inhibitor of proliferation (Pitsch, R. J. (1996), J. Vasc. Surg., 23:783-791; Horodyski, J. and Powell, R. J. (1996), J. Surg. Res., 66:115-118) prior to scraping. Cultures in which DNAzyme was absent repopulated the entire denuded zone within 3 days. ED5 inhibited this reparative response to injury and prevented additional growth in this area even after 6 days (data not shown). That ED5SCR had no effect in this system further demonstrates sequence-specific inhibition by ED5.

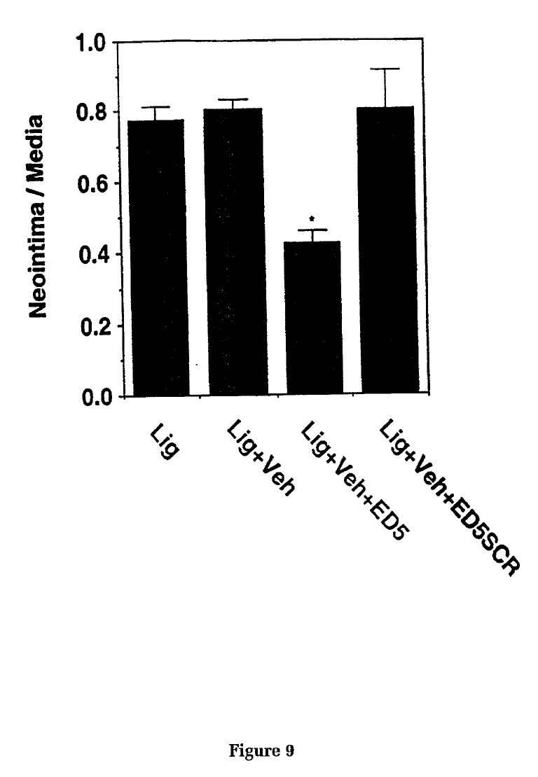

The effect of ED5 on neointima formation was investigated in a rat model. Complete ligation of the right common carotid artery proximal to the bifurcation results in migration of SMCs from the media to the intima where proliferation eventually leads to the formation of a neointima (Kumar, A. and Lindner, V. (1997), Arterioscl. Thromb. Vasc. Biol., 17:2238-2244; Bhawan, J. et al., (1977), Am. J. Pathol., 88:355-380; Buck, R. C. (1961), Circ. Res., 9:418-426). Intimal thickening 18 days after ligation was inhibited 50% by ED5 (FIG. 9). In contrast, neither its scrambled counterpart (FIG. 9) nor the vehicle control (FIG. 9) had any effect on neointima formation. These findings demonstrate the capacity of ED5 to suppress SMC accumulation in the vascular lumen in a specific manner, and argue against inhibition as a mere consequence of a “mass effect” (Kitze, B. (1998), Clin. Exp. Immunol., 111:278-285; Tharlow, R. J. and Hill, D. R. et al., (1996), Brit. J. Pharmacol., 118:457-465). Sequence specific inhibition of inducible NGFI-A protein expression and intimal thickening by EDS was also observed in the rat carotid balloon injury model (Santiago et al., 1999).

Further experiments revealed the capacity of hEDS to cleave (human) EGR-1 RNA. hED5 cleaved its substrate in a dose-dependent manner over a wide range of stoichiometric ratios. hED5 also cleaved in a time-dependent manner, whereas hED5SCR, its scrambled counterpart, had no such catalytic property (data not shown).

The specific, growth-inhibitory properties of antisense EGR-1 strategies reported herein suggest that EGR-1 inhibitors may be useful as therapeutic tools in the treatment of vascular disorders involving inappropriate SMC growth, endothelial growth and tumour growth.

### Example 3

Use of DNAzymes to Inhibit Growth of Malignant Cells

Materials and Methods

HepG2 cells were routinely grown in DMEM, pH 7.4, containing 10% fetal calf serum supplemented with antibiotics. The cells were trypsinized, resuspended in growth medium (to 10,000 cells/200 μl) and 200 μl transferred into sterile 96 well titre plates. Two days subsequently, 180 μl of the culture supernatant was removed, the cells were washed with PBS, pH 7.4, and refed with 180 μl of serum free media. After 6 h, the first transfection of DNAzyme (2 μg/200 μl wall, 0.75 μM final) was performed in tubes containing serum free media using FuGENE6 at a ratio of 1:3 (μg:μl). After 15 min incubation at room temperature, 180 μl of the culture supernantant was replaced with 180 μl of the transfection mix, After 24 h, 180 μl of the supernatant was replaced with 180 μl of new transfection mix, but this time in 5% FBS media. After 3 days, the cells were washed in PBS, pH 7.4, and resuspended by trypsinization in 100 μl trypsin-EDTA. The cells were shaken for approximately 5 min to ensure the cells were in suspension. The entire suspension was placed into 10 ml of Isoton II. That all the cells were transferred was ensured by pipetting Isoton II solution from tubes back into wells several times. Using Isoton II only, background cell number was determined. Each sample was counted three times and used to calculate mean counts and standard errors of each mean.

Results and Discussion

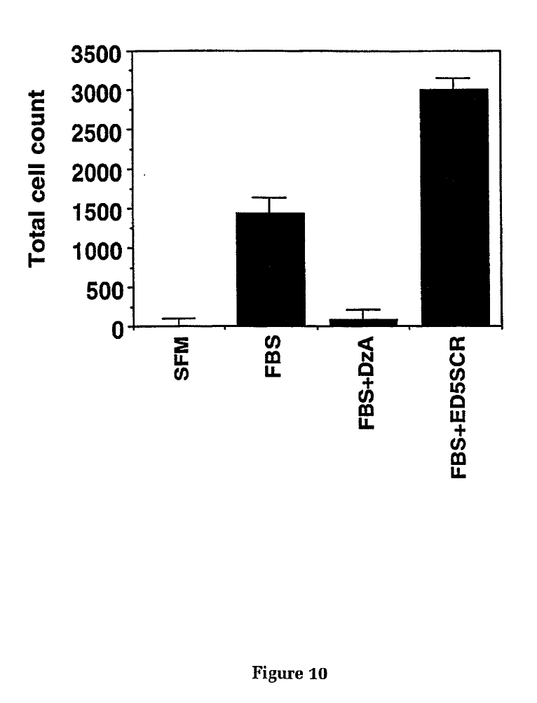

Our results indicate that serum stimulated HepG2 cell proliferation after 3 days (FIG. 10). Proliferation was almost completely suppressed by 0.75 μM of DzA (5′-caggggacaGGCTAGCTACAACGAcgttgcggg (SEQ ID NO:3), catalytic moiety in capitals), a DNAzyme targeting human EGR-1 mRNA (arms hybridize to nts 189-207) (FIG. 10). In contrast, HepG2 cell growth was not inhibited by ED5SCR (FIG. 10). Western blot analysis revealed that DzA strongly inhibited EGR-1 expression in HepG2 cells, whereas a size matched DNAzyme with different sequence (5′-tcagctgcaGGCTAGCTACAACGActcggcctt) (SEQ ID NO:24) had no effect (data not shown). These data indicate that inducible proliferation of this model human malignant cell line can be blocked by the EGR-1 DNAzyme. These findings suggest that EGR inhibitors may be clinically useful in therapeutic strategies targeting human cancer.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive. Throughout this application, various publications are cited. The disclosure of these publications is hereby incorporated by reference into this application to describe more fully the state of the art to which the invention pertains.

1. A method for the treatment of a tumour, the method comprising administering to a subject in need thereof an agent which inhibits induction of an EGR, an agent which decreases expression of an EGR or an agent which decreases the nuclear accumulation or activity of an EGR.

2. A method as claimed in claim 1 in which the agent inhibits angiogenesis.

3. A method as claimed in claim 1 in which the agent directly inhibits proliferation of the tumour cells.

4. A method as claimed in claim 2 in which the agent directly inhibits proliferation of the tumour cells.

5. A method as claimed in claim 1 in which the tumour is a solid tumour.

6. A method as claimed in claim 2 in which the tumour is a solid tumour.

7. A method as claimed in claim 4 in which the tumour is a solid tumour.

8. A method as claimed in claim 1 in which the EGR is EGR-1.

9. A method as claimed in claim 2 in which the EGR is EGR-1.

10. A method as claimed in claim 4 in which the EGR is EGR-1.

11. A method as claimed in claim 1 in which the expression of EGR is decreased.

12. A method as claimed in claim 2 in which the expression of EGR is decreased.

13. A method as claimed in claim 4 in which the expression of EGR is decreased.

14. A method as claimed in claim 5 in which the expression of EGR is decreased.

15. A method as claimed in claim 6 in which the expression of EGR is decreased by the use of an EGR antisense oligonucleotide.

16. A method as claimed in claim 7 in which the antisense oligonucleotide has a sequence selected from the group consisting of:
(i) ACA CTT TTG TCT GCT (SEQ ID NO:4), and
(ii) CTT GGC CGC TGC CAT (SEQ ID NO:2).

17. A method as claimed in claim 6 in which the expression of EGR is decreased by the cleavage of EGR mRNA by a sequence-specific ribozyme.

18. A method as claimed in claim 6 in which the expression of EGR is decreased by the use of a ssDNA targeted against EGR dsDNA the ssDNA molecule being selected so as to form a triple helix with the dsDNA.

19. A method as claimed claim 6 in which the expression of EGR is decreased by inhibiting transcription of the EGR gene using a nucleic acid transcriptional decoy.

20. A method as claimed in claim 6 in which the expression of EGR is decreased by the expression of antisense EGR mRNA.

21. A method as claimed in claim 6 in which the expression of EGR is decreased by cleavage of EGR mRNA by a sequence specific DNAzyme.

22. A method as claimed in claim 13 in which the DNAzyme comprises:
(i) a catalytic domain which cleaves mRNA at a purine:pyrimidine cleavage site;
(ii) a first binding domain contiguous with the 5′end of the catalytic domain; and
(iii) a second binding domain contiguous with the 3′ end of the catalytic domain,
wherein the binding domains are sufficiently complementary to two regions immediately flanking a purine:pyrimidine cleavage site within the region of EGR mRNA corresponding to nucleotides 168 to 332 as shown in SEQ ID NO:15, such that the DNAzyme cleaves the EGR mRNA.

23. A method as claimed in claim 13 in which the catalytic domain has the nucleotide sequence GGCTAGCTACAACGA.

24. A method as claimed in claim 13 in which the cleavage site is selected from the group consisting of:
(i) the GU site corresponding to nucleotides 198-199;
(ii) the GU site corresponding to nucleotides 200-201;
(iii) the GU site corresponding to nucleotides 264-265;
(iv) the AU site corresponding to nucleotides 271-272;
(v) the AU site corresponding to nucleotides 301-302;
(vi) the GU site corresponding to nucleotides 303-304; and
(vii) the AU site corresponding to nucleotides 316-317.

25. A method as claimed in claim 16 in which the cleavage site is the GU site corresponding to nucleotides 198-199, the AU site corresponding to nucleotides 271-272 or the AU site corresponding to nucleotides 301-302.

26. A method as claimed in claim 16 in which the DNAzyme has a sequence selected from the group consisting of:
(i) 5′-caggggacaGGCTAGCTACAACGAcgttgcggg (SEQ ID NO: 3);
(ii) 5′-tgcaggggaGGCTAGCTACAACGAaccgttgcg (SEQ ID NO:6);
(iii) 5′-catcctggaGGCTAGCTACAACGAgagcaggct (SEQ ID NO:7);
(iv) 5′-ccgcggccaGGCTAGCTACAACGAcctggacga (SEQ ID NO:8);
(v) 5′-ccgctgccaGGCTAGCTACAACGAcccggacgt (SEQ ID NO:9);
(vi) 5′-gcggggacaGGCTAGCTACAACGAcagctgcat (SEQ ID NO:10);
(vii) 5′-cagcggggaGGCTAGCTACAACGAatcagctgc (SEQ ID NO:11); and
(viii) 5′-ggtcagagaGGCTAGCTACAACGActgcagcgg (SEQ ID NO: 12).

27. A method as claimed in claim 18 in which the DNAzyme has the sequence 5′-caggggacaGGCTAGCTACAACGAcgttgcggg (SEQ ID NO:3).

28. A method as claimed in claim 18 in which the DNAzyme has the sequence 5′gcggggacaGGCTAGCTACAACGAcagctgcat (SEQ ID NO:10).

29. A method as claimed in claim 18 in which the DNAzyme has the sequence 5′-ccgcggccaGGCTAGCTACAACGAcctggacga (SEQ ID NO:8.

30. A method as claimed in claim 18 in which the DNAzyme has the sequence 5′-ccgctgccaGGCTAGCTACAACGAcccggacgt (SEQ ID NO:9).

31. A method as claimed in any one of claims 13 to 19, wherein the 3′-end nucleotide residue of the DNAzyme is inverted in the binding domain contiguous with the 3′ end of the catalytic domain.

32. A method as claimed in any one of claims 1 to 20 which further comprises administering one or more additional anti-cancer agents.

33. A method as claimed in claim 21 which further comprises administering one or more additional anti-cancer agents.

34. A method for inhibiting the growth or proliferation of a tumour cell, the method comprising contacting a tumour cell with an agent which inhibits induction of EGR, an agent which decreases expression of EGR or an agent which decreases the nuclear accumulation or activity of EGR.

35. A tumour cell which has been transformed by introducing into the call a nucleic acid molecule, the nucleic acid molecule comprising or encoding (i) an agent which inhibits induction of EGR, (ii) an agent which decreases expression of EGR, or (iii) an agent which decreases the nuclear accumulation or activity of EGR.

36. A method of screening for an agent which inhibits angiogenesis, the method comprising testing a putative agent for the ability to inhibit induction of EGR, decrease expression of EGR or decrease the nuclear accumulation or activity of EGR.
